(19) United States
(12) Patent Application Publication
(10) Pub. No.: US 2026/0125638 A1
(43) Pub. Date: May 7, 2026

(54) SYSTEMS AND METHODS FOR REMOVING CELLS FROM FLUIDIC DEVICES

(71) Applicant: Cellanome, Inc., Foster City, CA (US)

(72) Inventors: Nirvan ROUZBEH, Mountain View, CA (US); Elaheh FARJAMI, Santa Clara, CA (US); Meng TAING, Hayward, CA (US); Tarun KHURANA, Fremont, CA (US); Mostafa RONAGHI, Atherton, CA (US); Seyedsina MOEINZADEH, Mountain View, CA (US)

(21) Appl. No.: 19/376,703

(22) Filed: Oct. 31, 2025

Related U.S. Application Data
(60) Provisional application No. 63/715,354, filed on Nov. 1, 2024.

(51) Int. Cl. C12N 5/00

(52) U.S. Cl. CPC C12N 5/0081

(57) ABSTRACT

Disclosed herein are systems and methods for separating cells by selectively removing subpopulations of cells from fluidic devices. The methods can include synthesizing chambers that enclose a first subset of cells within a fluidic device and selectively removing a second subset of non-enclosed cells from the fluidic device.

### CROSS-REFERENCE

This application claims the benefit of U.S. Provisional Application No. 63/715,354 filed Nov. 1, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 21, 2025, is named 59528-734_201_SL.xml and is 9,811 bytes in size.

### BACKGROUND

Intercellular interactions pose numerous challenges in single cell assays. Cells secrete and sense complex consortias of proteins, nucleic acids, metabolites, vesicles, and ions that reflect their phenotype, health and environment. These secretions can propagate responses across cell populations. Even when cells are separated, soluble factors secreted by cells may diffuse to and influence the phenotypes, viability and behavior of nearby cells. For example, oxidative and heat stress responses by individual cells can manifest in population-wide phenotypic changes. Thus, the presence of nontarget bystander cells can impact the quality and repeatability of single cell analyses.

### SUMMARY

In one embodiment, the present application provides a method for separating a plurality of cells comprising inputting the plurality of cells into a channel of a fluidic device; synthesizing one or more chambers in the channel to enclose a first cell of the plurality of cells and not enclose a second cell of the plurality of cells; and removing the second cell from the channel, wherein the removing comprises contacting the second cell with a cell detachment reagent that detaches the second cell from a first surface of the channel or from a cell adherent support coupled to the first surface of the channel, and wherein the first cell remains enclosed in the one or more chambers during the removing.

In one aspect, prior to the removing, the second cell is adhered to the first surface of the channel or to the cell adherent support coupled to the first surface of the channel. In some such aspects, the contacting causes the second cell to detach from the first surface of the channel or from the cell adherent support coupled to the first surface of the channel. In another aspect, the removing causes the first cell to become suspended within a chamber of the one or more chambers. In an additional aspect, the method further comprises incubating the first cell following the removing, wherein during the incubating, the first cell binds or adheres to the first surface of the channel or to the cell adherent support coupled to the first surface of the channel.

In certain aspects, the one or more chambers co-enclose a capture element with the first cell. In particular aspects, the one or more chambers co-enclose the capture element and the cell adherent support with the first cell, and: the cell adherent support is coupled to the first surface of the channel and the capture element is coupled to a second surface of the channel opposite the first surface of the channel; or the cell adherent support and the capture element are coupled to the first surface of the channel. In further aspects, the cell adherent support is adjacent to the capture element on the first surface of the channel. In another aspect, the capture element comprises an oligonucleotide. In one such aspect, the oligonucleotide comprises an mRNA capture sequence.

In another embodiment, the present disclosure provides a method for separating a plurality of cells comprising: inputting the plurality of cells into a channel of a fluidic device; contacting the plurality of cells with an adhesion inhibitor that diminishes a binding affinity of at least a subset of the plurality of cells to a first surface of the channel or a cell adherent support coupled to the first surface of the channel; synthesizing one or more chambers in the channel to enclose a first cell of the plurality of cells and not enclose a second cell of the plurality of cells; and removing the second cell from the channel, wherein the first cell remains enclosed in the one or more chambers during the removing.

In a certain aspect, the contacting is prior to the inputting; the contacting is subsequent to the inputting; or the inputting comprises loading the adhesion inhibitor into the channel with the plurality of cells. In a further aspect, the adhesion inhibitor is loaded into the channel with one or more polymer precursors of the one or more chambers, and the synthesizing comprises forming one or more walls of the one or more chambers using the one or more polymer precursors. In an additional aspect, the adhesion inhibitor diminishes an integrin binding affinity of the plurality of cells of the subset of the plurality of cells. In another aspect, the adhesion inhibitor binds to the plurality of cells or the subset of the plurality of cells. In a particular aspect, the adhesion inhibitor comprises an antibody, actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof.

In an additional aspect, the adhesion inhibitor comprises a metal chelator. In one such aspect, the metal chelator comprises a formation constant of at least about 10⁸ for Ca²⁺, Mg²⁺, and Mn²⁺. In specific aspects, the metal chelator comprises EDTA, glutamic acid diacetate (GLDA), dicarboxymethyl alaninate, glutamate diacetate, nitrilotriacetic acid (NTA), 1,4,7,10-tetraazacyclododecane-1,4,7,10-tetraacetic acid, (DOTA), diethylenetriaminepentaacetic acid (DTPA), 1,4-bis(carboxymethyl)-6-[bis(carboxymethyl)]amino-6-methylperhydro-1,4-diazepine (AAZTA), 2,2′-(6-((carboxymethyl)amino)-1,4-diazepane-1,4-diyl)diacetic acid (DATA), N,N′-bis(2-hydroxybenzyl)-ethylenediamine-N,N′-diacetic acid, (HBED), bis(2-pyridylcarbonyl) amine (BPCA), 4-acetylamino-4-[2-[(3-hydroxy-1,6-dimethyl-4-oxo-1,4-dihydro-pyridin-2-ylmethyl)-carbamoyl]-ethyl]-heptanedioicacid bis-[(3-hydroxy-1,6-dimethyl-4-oxo-1,4-dihydro-pyridin-2-ylmethyl)-amide](CP256), desferrioxamine B (DFO), 1,4,7,10,13,16-hexaazacyclohexadecane-N,N′,N″,N′″,N″″,N′″″-hexaacetic acid (HEHA), {4-[2-(bis-carboxymethylamino)-ethyl]-7-carboxymethyl-[1,4,7]triazonan-1-yl}-acetic acid (NETA), 1,4,7-triazacyclononane-1,4-bis[methylene(hydroxymethyl)phosphinic acid]-7-[methylene(2-carboxyethyl)phosphinic acid](NOPO), 1,4,7-triazacyclononane-1,4,7-triacetic acid, (NOTA), 3,6,9,15-tetraazabicyclo[9.3.1]-pentadeca-1(15),11,13-triene-3,6,9,-triacetic acid (PCTA), 1,4,7,10,13-pentaazacyclopentadecane-N,N′,N″,N′″,N″″-pentaacetic acid (PEPA), N,N′-bis(2-hydroxy-5-sulfobenzyl)-ethylenediamine-N,N′-diacetic acid (SHBED), N,N′,N″, tris(2-mercaptoethyl)-1,4,7-triazacyclononane (TACN-TM), 1,4,8,11-tetraazacyclotetradecane-1,4,8,11-tetraacetic acid, (TETA), 1,4,7-triazacyclononane-1,4,7-tris[methyl(2-carboxyethyl)phosphinic acid](TRAP), or a combination thereof.

In some aspects, the method further comprises releasing the adhesion inhibitor from the first cell. In particular aspects, the releasing comprises contacting the first cell with a cell detachment reagent, sonicating the fluidic device, ultrasonicating the fluidic device, or a combination thereof.

In yet another embodiment, the present disclosure provides a method for separating a plurality of cells comprising: inputting the plurality of cells into a channel of a fluidic device comprising a first surface comprising a plurality of oligonucleotides coupled to the first surface, wherein a first cell of the plurality of cells associates with a first subset of oligonucleotides of the plurality of oligonucleotides; synthesizing one or more chambers in the channel to co-enclose the first cell with the first subset of oligonucleotides and not enclose a second cell of the plurality of cells; and removing the second cell from the channel, wherein the first cell remains enclosed in the one or more chambers during the removing.

In certain aspects, the removing comprises contacting the second cell with a cell detachment reagent. In select aspects, the second cell is associated with a second subset of oligonucleotides of the plurality of oligonucleotides that are not enclosed within the one or more chambers, and the removing comprises cleaving the second subset of the oligonucleotides from the first surface. In specific aspects, the cleaving comprises chemical cleavage, enzymatic cleavage, or photolysis.

In some aspects, the associating comprises intercalating into, partitioning onto, or binding to a terminus portion of one or more oligonucleotides of the plurality of oligonucleotides. In certain aspects, the second cell does not associate with the plurality of oligonucleotides.

In a further embodiment, the present disclosure provides a method for separating a plurality of cells comprising: inputting the plurality of cells into a channel of a fluidic device comprising a first surface comprising a plurality of oligonucleotides coupled to the first surface and a cell adherent support coupled to the plurality of oligonucleotides, wherein a first cell of the plurality of cells binds to the cell adherent support; synthesizing one or more chambers in the channel to co-enclose the first cell with a first subset of oligonucleotides of the plurality of oligonucleotides and not enclose a second cell of the plurality of cells, and removing the second cell from the channel, wherein the removing comprises contacting the second cell with a cell detachment reagent, and wherein the first cell remains enclosed in the one or more chambers during the removing.

In certain aspects, prior to the removing, the second cell is associated with a second subset of the plurality of oligonucleotides or is bound to the cell adherent support coupled to the second subset of the plurality of oligonucleotides. In other aspects, the second cell does not associate with oligonucleotides of the plurality of oligonucleotides or bind to the cell adherent support coupled to the plurality of oligonucleotides.

In another embodiment, the present disclosure provides a method for separating a plurality of cells comprising: inputting the plurality of cells into a channel of a fluidic device comprising a first surface comprising a plurality of oligonucleotides coupled to the first surface and a cell adherent support coupled to the plurality of oligonucleotides, wherein a first cell of the plurality of cells binds to the cell adherent support of a first subset of the plurality of oligonucleotides; synthesizing one or more chambers in the channel to co-enclose the first cell with the first subset of the oligonucleotides of the plurality of oligonucleotides and not enclose a second cell of the plurality of cells that is bound to the cell adherent support of a second subset of the plurality of oligonucleotides, and removing the second cell from the channel, wherein the removing comprises: cleaving the second subset of the oligonucleotides from the first surface, cleaving the cell adherent support from the second subset of the plurality of oligonucleotides, or cleaving the second subset of the plurality of oligonucleotides from the first surface and cleaving the cell adherent support from the second subset of the plurality of oligonucleotides; and wherein the first cell remains enclosed in the one or more chambers during the removing.

In some aspects, the cleaving comprises photocleavage, chemical cleavage, thermal cleavage, pH-mediated cleavage, or enzymatic cleavage. In a select aspect, the photocleavage comprises ultraviolet (UV) light. In a particular aspect, the second cell is adhered to the first surface or to an additional cell adherent support coupled to the first surface prior to the removing, and wherein the removing comprises detaching the second cell from the first surface or from the additional cell adherent support coupled to the first surface.

In an additional embodiment, the present disclosure provides a method for separating a plurality of cells comprising: inputting the plurality of cells into a channel of a fluidic device, wherein the channel comprises a first surface comprising: (a) a cell adherent support, and (b) a non-adherent support to which the plurality of cells comprise a diminished binding affinity relative to the cell adherent support; synthesizing one or more chambers in the channel to co-enclose a first cell of the plurality of cells with the cell adherent support and not enclose a second cell of the plurality of cells that is disposed above, below, or on the non-adherent support; and removing the second cell from the channel; wherein the first cell remains enclosed in the one or more chambers during the removing.

In particular aspects, the plurality of cells comprise a diminished binding affinity to the non-adherent support relative to the first surface. In further aspects, the cell adherent support and non-adherent support are patterned over at least a portion of the first surface. In one such aspect, the channel comprises a first plurality of regions comprising the cell adherent support and a second plurality of regions comprising the non-adherent support. In a further aspect, the first and second plurality of regions do not overlap.

In certain aspects, the non-adherent support comprises a synthetic polymer. In additional aspects, the non-adherent support comprises an acrylate, an acrylamide, an amine, a silane, a PEG, a PEG-silane, an acrylate-PEG-silane, a silane-PEG-silane, a PEG-amine, an acrylamide-PEG-amine, acrylate-PEG-amine, a COOH-PEG-amine, or a combination thereof. In particular aspects, the non-adherent support is disposed outside of the one or more chambers. In select aspects, the removing comprises detaching the second cells from the non-adherent support.

In further embodiments, the present disclosure provides a method for separating a plurality of cells comprising: inputting the plurality of cells into a channel of a fluidic device; synthesizing one or more chambers in the channel to enclose a first cell of the plurality of cells and not enclose a second cell of the plurality of cells; irradiating the second cell with light; and removing the second cell from the channel; wherein the first cell remains in the one or more chambers following the removing.

In an aspect, the first cell is not irradiated with the light. In another aspect, the light comprises ultraviolet (UV) light. In particular aspects, the light kills the second cell. In certain aspects, the light detaches the second cell from a surface of the channel. In specific aspects, the light diminishes an affinity of the second cell for a surface of the channel or an adherent support coupled to the surface of the channel.

In an additional embodiment, the present disclosure provides a method for separating a plurality of cells comprising: inputting the plurality of cells into a channel of a fluidic device comprising a first surface comprising a cell adherent support; synthesizing one or more chambers in the channel to enclose a first cell of the plurality of cells and not enclose a second cell of the plurality of cells, wherein the second cell is bound to a first portion of the cell adherent support; detaching at least the first portion of the cell adherent support from the first surface of the channel, thereby detaching the second cell from the surface of the channel; and removing the second cell from the channel; wherein the first cell remains enclosed in the one or more chambers during the removing.

In certain aspects, a second portion of the cell adherent support is co-enclosed within the one or more chambers with the first cell. In further aspects, the detaching does not detach the second portion of the cell adherent support from the first surface of the channel. In particular aspects, the detaching comprises contacting the cell adherent support or the first portion of the cell adherent support with a polyanion. In a select aspect, the polyanion displaces at least a portion of the cell adherent support and then the polyanion is coupled to the first surface. In an additional aspect, the polyanion couples to the first surface after the detaching at least the first portion of the cell adherent support from the first surface. In a particular, the polyanion comprises polyacrylic acid, polymethacrylic acid, alginate, heparan sulfate, polyglutamic acid, polyaspartic acid, poly(4-vinylbenzoic acid), hyaluronic acid, polystyrenesulfonate, heparin, chondroitin sulfate, dextran sulfate, polymethacrylic acid, carboxymethyl cellulose, polystyrene sulfonate, DNA, RNA, a copolymer thereof, or a combination thereof.

In some aspects, the polyanion comprises a molecular weight of about 0.5 to 1 kilodaltons (kDa), about 0.5 to 2.5 kDa, about 0.5 to 5 kDa, about 0.5 to 10 kDa, about 0.5 to 25 kDa, about 0.5 to 50 kDa, about 0.5 to 100 kDa, about 0.5 to 250 kDa, about 0.5 to 500 kDa, about 0.5 to 1000 kDa, about 0.5 to 2500 kDa, about 0.5 to 5000 kDa, about 0.5 to 10000 kDa, about 1 to 2.5 kDa, about 1 to 5 kDa, about 1 to 10 kDa, about 1 to 25 kDa, about 1 to 50 kDa, about 1 to 100 kDa, about 1 to 250 kDa, about 1 to 500 kDa, about 1 to 1000 kDa, about 1 to 2500 kDa, about 1 to 5000 kDa, about 1 to 10000 kDa, about 2.5 to 5 kDa, about 2.5 to 10 kDa, about 2.5 to 25 kDa, about 2.5 to 50 kDa, about 2.5 to 100 kDa, about 2.5 to 250 kDa, about 2.5 to 500 kDa, about 2.5 to 1000 kDa, about 2.5 to 2500 kDa, about 2.5 to 5000 kDa, about 2.5 to 10000 kDa, about 5 to 10 kDa, about 5 to 25 kDa, about 5 to 50 kDa, about 5 to 100 kDa, about 5 to 250 kDa, about 5 to 500 kDa, about 5 to 1000 kDa, about 5 to 2500 kDa, about 5 to 5000 kDa, about 5 to 10000 kDa, about 10 to 25 kDa, about 10 to 50 kDa, about 10 to 100 kDa, about 10 to 250 kDa, about 10 to 500 kDa, about 10 to 1000 kDa, about 10 to 2500 kDa, about 10 to 5000 kDa, about 10 to 10000 kDa, about 25 to 50 kDa, about 25 to 100 kDa, about 25 to 250 kDa, about 25 to 500 kDa, about 25 to 1000 kDa, about 25 to 2500 kDa, about 25 to 5000 kDa, about 25 to 10000 kDa, about 50 to 100 kDa, about 50 to 250 kDa, about 50 to 500 kDa, about 50 to 1000 kDa, about 50 to 2500 kDa, about 50 to 5000 kDa, about 50 to 10000 kDa, about 100 to 250 kDa, about 100 to 500 kDa, about 100 to 1000 kDa, about 100 to 2500 kDa, about 100 to 5000 kDa, about 100 to 10000 kDa, about 250 to 500 kDa, about 250 to 1000 kDa, about 250 to 2500 kDa, about 250 to 5000 kDa, about 250 to 10000 kDa, about 500 to 1000 kDa, about 500 to 2500 kDa, about 500 to 5000 kDa, about 500 to 10000 kDa, about 1000 to 2500 kDa, about 1000 to 5000 kDa, about 1000 to 10000 kDa, about 2500 to 5000 kDa, about 2500 to 10000 kDa, or about 5000 to 10000 kDa. In select aspects, the polyanion comprises a molecular weight of at least about 0.5 kDa, about 1 kDa, about 2.5 kDa, about 5 kDa, about 10 kDa, about 25 kDa, about 50 kDa, about 100 kDa, about 250 kDa, about 500 kDa, about 1,000 kDa, about 2500 kDa, or about 5000 kDa. In some cases, the polyanion comprises a molecular weight of at most about 1 kDa, about 2.5 kDa, about 5 kDa, about 10 kDa, about 25 kDa, about 50 kDa, about 100 kDa, about 250 kDa, about 500 kDa, about 1,000 kDa, about 2,500 kDa, about 5,000 kDa, or about 10,000 kDa. In additional aspects, the polyanion comprises a molecular weight of about 100 to 5000 kDa. In certain aspects, walls of the one or more chambers are impermeable to the polyanion.

In certain aspects, the cell adherent support comprises actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof. In particular aspects, the cell adherent support is directly coupled to the first surface of the channel. In additional aspects, the cell adherent support is coupled to a polymeric coating on the first surface of the channel. In one such aspect, the polymeric coating comprises poly-l-ornithine.

In select aspects, the removing comprises a wash step. In particular aspects, the removing comprises heating the channel of the fluidic device to between about 30-37° C. or 30-42° C. In some aspects, the removing comprises raising or lowering a pH by at least about 0.25, by about least about 0.5, by at least about 0.75, by at least about 1, by at least about 1.25, by at least about 1.5, or by at least about 2 within the fluidic device.

In a particular aspect, the wash step utilizes a solution that: i) comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM, less than about 10⁻⁴ mM, less than about 10⁻⁵ mM, or less than about 10⁻⁶ mM Ca²⁺; ii) comprises less than about less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM, less than about 10⁻⁴ mM, less than about 10⁻⁵ mM, or less than about 10⁻⁶ mM Mg²⁺; comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM, less than about 10⁻⁴ mM, less than about 10⁻⁵ mM, or less than about 10⁻⁶ mM Mn²⁺; comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM, less than about 10⁻⁴ mM, less than about 10⁻⁵ mM, or less than about 10⁻⁶ mM total divalent cations; is essentially free of Ca²⁺; is essentially free of Mg²⁺; is essentially free of Mn²⁺; is essentially free of divalent cations; comprises a metal chelator; or a combination thereof.

In some aspects, the removing comprises sonication. In one such aspect, the sonicating comprises: a frequency of about 0.02 to 0.25, about 0.02 to 0.5, about 0.02 to 1, about 0.02 to 10, about 0.02 to 25, about 0.02 to 50, about 0.02 to 100, about 0.02 to 250, about 0.02 to 500, about 0.02 to 1000, about 0.25 to 1, about 0.25 to 2.5, about 0.25 to 5, about 0.25 to 10, about 0.25 to 25, about 0.25 to 50, about 0.25 to 100, about 0.25 to 250, about 0.25 to 500, about 0.25 to 1000, about 1 to 10, about 1 to 25, about 1 to 50, about 1 to 100, about 1 to 200, about 1 to 500, about 1 to 1000, about 5 to 25, about 5 to 50, about 5 to 100, about 5 to 200, about 5 to 500, about 5 to 1000, about 10 to 25, about 10 to 50, about 10 to 100, about 10 to 200, about 10 to 500, about 10 to 1000, about 25 to 50, about 25 to 100, about 25 to 200, about 25 to 500, about 25 to 1000, about 50 to 100, about 50 to 200, about 50 to 500, about 50 to 1000, about 100 to 200, about 100 to 500, or about 100 to 1000 kilohertz (kHz); a duration of about 0.25 to 1, about 0.25 to 2.5, about 0.25 to 5, about 0.25 to 10, about 0.25 to 20, about 0.25 to 30, about 1 to 2.5, about 1 to 5, about 1 to 10, about 1 to 20, about 1 to 30, about 2.5 to 5, about 2.5 to 10, about 2.5 to 20, about 2.5 to 30, about 5 to 10, about 5 to 20, about 5 to 30, about 10 to 20, about 10 to 30, or about 20 to 30 minutes; an intensity of about 0.25 to 1, about 0.25 to 2.5, about 0.25 to 5, about 0.25 to 10, about 0.25 to 25, about 0.25 to 50, about 1 to 2.5, about 1 to 5, about 1 to 10, about 1 to 25, about 1 to 50, about 2.5 to 5, about 2.5 to 10, about 2.5 to 25, about 2.5 to 50, about 5 to 10, about 5 to 25, about 5 to 50, about 10 to 50, or about 25 to 50 W/cm²; or a combination thereof.

In certain aspects, the removing comprises contacting the second cell with a cell detachment reagent. In some aspects, the cell detachment reagent comprises a protease, a metal chelator, a polyanion, or a combination thereof. In particular aspects, the cell detachment reagent comprises a protease, a metal chelator, or a combination thereof. In further aspects, the protease comprises accutase, dispase, chymotrypsin, collagenase, GluC endoprotease, pepsin, trypsin, TrypLE, or a combination thereof. In select aspects, the metal chelator comprises ethylenediaminetetraacetic acid (EDTA), glutamic acid diacetate (GLDA), dicarboxymethyl alaninate, glutamate diacetate, nitrilotriacetic acid (NTA), 1,4,7,10-tetraazacyclododecane-1,4,7,10-tetraacetic acid, (DOTA), diethylenetriaminepentaacetic acid (DTPA), 1,4-bis(carboxymethyl)-6-[bis(carboxymethyl)]amino-6-methylperhydro-1,4-diazepine (AAZTA), 2,2′-(6-((carboxymethyl)amino)-1,4-diazepane-1,4-diyl)diacetic acid (DATA), N,N′-bis(2-hydroxybenzyl)-ethylenediamine-N,N′-diacetic acid, (HBED), bis(2-pyridylcarbonyl) amine (BPCA), 4-acetylamino-4-[2-[(3-hydroxy-1,6-dimethyl-4-oxo-1,4-dihydro-pyridin-2-ylmethyl)-carbamoyl]-ethyl]-heptanedioicacid bis-[(3-hydroxy-1,6-dimethyl-4-oxo-1,4-dihydro-pyridin-2-ylmethyl)-amide](CP256), desferrioxamine B (DFO), 1,4,7,10,13,16-hexaazacyclohexadecane-N,N′,N″,N′″,N″″,N′″″-hexaacetic acid (HEHA), {4-[2-(bis-carboxymethylamino)-ethyl]-7-carboxymethyl-[1,4,7]triazonan-1-yl}-acetic acid (NETA), 1,4,7-triazacyclononane-1,4-bis[methylene(hydroxymethyl)phosphinic acid]-7-[methylene(2-carboxyethyl)phosphinic acid](NOPO), 1,4,7-triazacyclononane-1,4,7-triacetic acid, (NOTA), 3,6,9,15-tetraazabicyclo[9.3.1]-pentadeca-1(15),11,13-triene-3,6,9,-triacetic acid (PCTA), 1,4,7,10,13-pentaazacyclopentadecane-N,N′,N″,N′″,N″″-pentaacetic acid (PEPA), N,N′-bis(2-hydroxy-5-sulfobenzyl)-ethylenediamine-N,N′-diacetic acid (SHBED), N,N′,N″, tris(2-mercaptoethyl)-1,4,7-triazacyclononane (TACN-TM), 1,4,8,11-tetraazacyclotetradecane-1,4,8,11-tetraacetic acid, (TETA), 1,4,7-triazacyclononane-1,4,7-tris[methyl(2-carboxyethyl)phosphinic acid](TRAP), polyacrylic acid, alginate, or a combination thereof. In some such aspects, the polyacrylic acid, the alginate, or the combination thereof comprises a molecular weight of at least about 1 megadalton (MDa). In certain aspects, the metal chelator comprises a formation constant of greater than about 10⁸ for Ca²⁺, Mg²⁺, and Mn²⁺. In some aspects, the polyanion comprises polyacrylic acid, polymethacrylic acid, alginate, heparan sulfate, polyglutamic acid, polyaspartic acid, poly(4-vinylbenzoic acid), hyaluronic acid, polystyrenesulfonate, heparin, chondroitin sulfate, dextran sulfate, polymethacrylic acid, carboxymethyl cellulose, or a combination thereof.

In some aspects, the first cell is suspended prior to the removing. In certain aspects, the cell adherent support comprises actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof. In select aspects, the cell adherent peptide comprises a sequence selected from RGD, GFOGER (SEQ ID NO: 1), YIGSR (SEQ ID NO: 2), LRE, GRGDS (SEQ ID NO: 3), CKKQRFRHRNRKG (SEQ ID NO: 4), KRSR (SEQ ID NO: 5), VPGIG (SEQ ID NO: 6), MNYYSNS (SEQ ID NO: 7), CSVTCG (SEQ ID NO: 8), GFRGDGQ (SEQ ID NO: 9), HAV, FLPASGL (SEQ ID NO: 10), or a combination thereof.

In particular aspects, the method further comprises incubating the first cell within the one or more chambers following the removing the second cell from the channel. In some aspects, the first cell adheres to the cell adherent support during the incubating. In one aspect, the first cell comprises an adherent cell. In another aspect, the first cell comprises a non-adherent cell. In some aspects, the second cell comprises an adherent cell. In another aspect, the second cell comprises a non-adherent cell. In certain aspects, the first cell and the second cell comprise the same type of cell. In other aspects, the first cell is of a different type than the second cell. In particular aspects, the first cell, the second cell, or the first cell and the second cell comprise an adipocyte, a cardiomyocyte; chondrocyte, an ectoderm, an embryonic stem cell, an endodermal cell, an endothelial cell, a fibroblast, a hematopoietic cell, a hematopoietic stem cell, a satellite, a hepatocyte, an islet cell, a keratinocyte, a mesenchymal cell, a mesenchymal stem cell, a progenitor cell, a myoblast, a myocyte, a neural cell, an oligodendrocyte, an osteoblast, a pancreatic epithelial cell, a skeletal myocyte cell, a smooth muscle cell, or a white blood cell.

In certain aspects, the synthesizing comprises projecting light into the channel with a spatial energy modulating element such that the projected light causes cross-linking of one or more polymer precursors to at least partially form the one or more chambers. In some aspects, the one or more polymer precursors comprise a crosslinker. In select aspects, the crosslinker comprises a diacrylate, a dimethacrylate, a diacrylamide, a dimethacrylamide, a diolefin, a polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or a combination thereof. In additional aspects, the polymer precursor comprises a photoinitiator. In further aspects, the polymer precursor comprises a porogen.

In further aspects, the one or more chambers comprise a hydrogel. In specific aspects, the one or more chambers extend from a bottom surface of the channel to a top surface of the channel. In particular aspects, the method further comprises degrading a chamber of the one or more chambers.

In select aspects, the method further comprises determining a characteristic of the first cell.

In one such aspect, the characteristic of the first cell is a morphological feature. In particular aspects, the morphological feature is a diameter; largest dimension; area; perimeter; eccentricity; shape; color; spread; polarity; neurite, axon, and/or dendrite size, shape, distribution, or density; nucleus size or shape; vacuole size, shape, distribution, or density; granule size, shape, distribution, or density; or inclusion body size, shape, distribution, or density.

In another aspect, the characteristic of the first cell is a cell type. In some such aspects, the cell type is determined by measuring a fluorescence signal from an antibody bound to a surface marker of the first cell.

In an additional aspect, the characteristic comprises a soluble factor secreted by the first cell. In particular aspects, said detecting said soluble factor comprises: (i) disposing a capture surface comprising an affinity reagent that binds the soluble factor adjacent to the first cell, and (ii) detecting the soluble factor bound to the capture surface. In certain aspects, said detecting said soluble factor bound to said capture surface comprises: (i) contacting said soluble factor bound to said capture surface with a labeled antibody configured to bind to the soluble factor, and (ii) detecting the labeled antibody. In select aspects, the method further comprises removing the soluble factor from the channel and detecting a second soluble factor secreted by the first cell subsequent to said removing the soluble factor from the channel.

In particular aspects, the first cell comprises an effector cell, and wherein the characteristic of the first cell comprises cytotoxicity. In one such aspect, the one or more chambers enclose one or more target cells with the effector cell, and said measuring said cytotoxicity comprises counting dead and/or viable cells from among the one or more target cells.

In select aspects, the characteristic of the first cell comprises activation. In some such aspects, the activation is caused by: (i) contact between the first cell and a third cell of the plurality of cells; (ii) a soluble factor secreted by the second cell; (iii) a soluble factor secreted by the third cell; or (iv) a combination thereof. In certain aspects, (i) the third cell is co-enclosed with the first cell in the one or more chambers; or (ii) the first cell and the third cell are enclosed in different chambers of the one or more chambers. In some aspects, said determining said activation comprises detecting a surface marker, a morphology, a cytotoxicity, a proliferation rate, a soluble factor secreted by the first cell, a genomic sequence, an mRNA, or a combination thereof. In particular aspects, said detecting said surface marker comprises contacting the first cell with a binding agent configured to bind to the surface marker, and detecting the binding agent.

In certain aspects, the characteristic of the first cell is a guide ribonucleic acid (RNA) associated with a genetic modification of the cell. In select aspects, the guide RNA is coupled to an exogenous messenger RNA (mRNA). In additional aspects, the guide RNA is coupled to a barcode. In further aspects, the guide RNA is coupled to a polyA tail. In additional aspects, the method further comprises generating a cDNA molecule comprising a complement of the guide RNA sequence, the exogenous mRNA, the barcode, or a combination thereof. In particular aspects, the cDNA molecule is coupled to a spatial location tag corresponding to a unique location within the fluidic channel. In specific aspects, the generating the cDNA molecule comprises capturing the guide RNA on the capture probe comprising the spatial location tag or the complement thereof, and reverse transcribing the guide RNA on the capture probe, thereby generating the cDNA molecule. In select aspects, the method further comprises sequencing the cDNA, thereby detecting the guide RNA associated with the genetic modification of the cell.

In particular aspects, the characteristic is an mRNA expressed by the first cell. In some such aspects, the determining comprises lysing the cell, capturing the mRNA on a capture element coupled to a surface of the channel, reverse transcribing the mRNA to generate a cDNA molecule that optionally comprises a barcode sequence derived from the capture element, and sequencing the cDNA molecule.

Another embodiment of the present disclosure provides a fluidic device comprising a first surface comprising a first region comprising a cell adherent support and a second region comprising a non-adherent support. In one aspect, the cell adherent support comprises actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof. In another aspect, the second region is adjacent to the first region. In certain aspects, the non-adherent support comprises an acrylate, an acrylamide, an amine, a silane, a PEG, a PEG-silane, an acrylate-PEG-silane, a silane-PEG-silane, a PEG-amine, an acrylamide-PEG-amine, acrylate-PEG-amine, a COOH-PEG-amine, or a combination thereof. In particular aspects, the cell adherent support and non-adherent support are patterned over at least a portion of the first surface. In specific aspects, the first surface comprises a plurality of instances of the first region and a plurality of instances of the second region. In select aspects, the plurality of instances of the first region and the plurality of instances of the second region are tessellated over at least a portion of the first surface.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic illustration of a portion of a channel disposed in a fluidic device, according to some embodiments.

FIG. 2A is an illustration of a portion of a system as provided herein including an energy source, according to some embodiments.

FIG. 2B is an illustration of a polymer matrix being formed around a biological component in a portion of a system as provided herein, according to some embodiments;

FIG. 2C is an illustration of a method of forming a polymer matrix around a biological component in a system as provided herein, according to some embodiments;

FIG. 3A is an illustration of a top view of the bottom layer of a flow cell without any spatial barcoded oligonucleotides.

FIG. 3B is an illustration of a top view of the spacer layer with a cut-out region suitable for use as part of the flow cell.

FIG. 3C is an illustration of a top view of the top layer of the flow cell where the top layer has an inlet and outlet opening.

FIG. 3D is an illustration of a cross-sectional side view of a top layer and a bottom layer sandwiching a spacer layer to form multiple channels of a flow cell.

FIG. 4A is an illustration of a system with a flow cell and an imaging apparatus that are capable of implementing methods of the present disclosure.

FIG. 4B is an illustration of a flow cell, including a blown-up view of a portion of a channel containing cells disposed in hydrogel chambers.

FIG. 5 is a schematic of a computer system 1501 that may be programmed or otherwise configured to perform methods described herein.

FIG. 6 is a schematic of a method for separating cells by synthesizing chambers at least partially enclosing a subset of the cells and removing cells that are not at least partially enclosed by the chambers.

FIG. 7 is a schematic of a method for separating cells that includes synthesizing chambers at least partially enclosing a subset of the cells and then contacting the cells with a cell detachment reagent to remove cells that are not at least partially enclosed by the chambers.

FIG. 8 is a schematic of a method for separating cells that includes contacting the cells with an adhesion inhibitor, at least partially enclosing a subset of the cells in chambers, and removing the cells not at least partially enclosed by the chambers.

FIG. 9 is a schematic of a method for separating cells that includes allowing a subset of the cells to bind to surface-bound oligonucleotides, at least partially enclosing the subset of cells within chambers, and removing non-enclosed cells.

FIG. 10 is a schematic of a method for separating cells that includes enclosing a first subset of cells adjacent to an adherent support, not enclosing a second subset of cells that are adjacent to a non-adherent support, and removing the second subset of cells.

FIGS. 11A-11D are images of fibroblasts in a flow cell channel. FIG. 11A is an image of the flow cell channel following chamber synthesis around a subset of the fibroblasts. FIG. 11B is an image of the flow cell channel following a buffer wash step. FIG. 11C is an image of the flow cell channel following EDTA and buffer washes. FIG. 11D is an image of the flow cell channel following an incubation period.

FIG. 12 is a schematic of a method for separating cells that includes allowing a subset of the cells to bind to an adherent support, at least partially co-enclosing the cells with surface-bound oligonucleotide barcodes in chambers, and removing cells that are not at least partially enclosed by the chambers.

FIG. 13 is a schematic of a method for separating cells wherein a subset of cells are detached from a fluidic device surface with light.

FIG. 14 is a schematic of a method for separating cells that comprises detaching at least a portion of a cell and/or a cell adherent support and cell from a fluidic device surface.

FIG. 15A is a brightfield image of a field of view within a fluidic device following chamber synthesis.

FIG. 15B is a brightfield image of the field-of-view of the fluidic device shown in FIG. 15A following a buffer wash step.

FIG. 16A is a brightfield image of a field of view within a fluidic device following chamber synthesis.

FIG. 16B is a brightfield image of the field-of-view of the fluidic device shown in FIG. 16A following a wash step with a cell detachment reagent.

FIG. 17 is a brightfield image of a portion of a fluidic device surface that includes (1) cell-containing, adherent support-coated regions and (2) non-adherent support-coated regions.

### DETAILED DESCRIPTION

Disclosed herein are methods for separating collections of cells in fluidic devices, and for removing subpopulations of cells that were not selected for analysis. Inefficient nontarget cell removal often limits the accuracy and repeatability of cell separation and analysis methods. Following target cell collection, for example through antibody capture or enclosure within a hydrogel chamber, non-collected cells are typically removed through wash steps. However, some cells resist removal by adhering to a surface or associating with a fluidic device-bound capture agent or structure. For example, many cells adhere to glass surfaces. Curtis, J Cell Biol., 1964; 20(2):199-215. This problem is often pronounced in complex biological samples that include multiple cell types. For example, peripheral blood mononuclear cell (PBMC) preparations include diverse populations of immune cells with disparate surface adherence mechanisms. Separating individual cells from PBMCs thus requires efficient means for selecting target cell types and removing nontarget cells. The present disclosure addresses this need by providing systems and methods for efficient cell removal and separation.

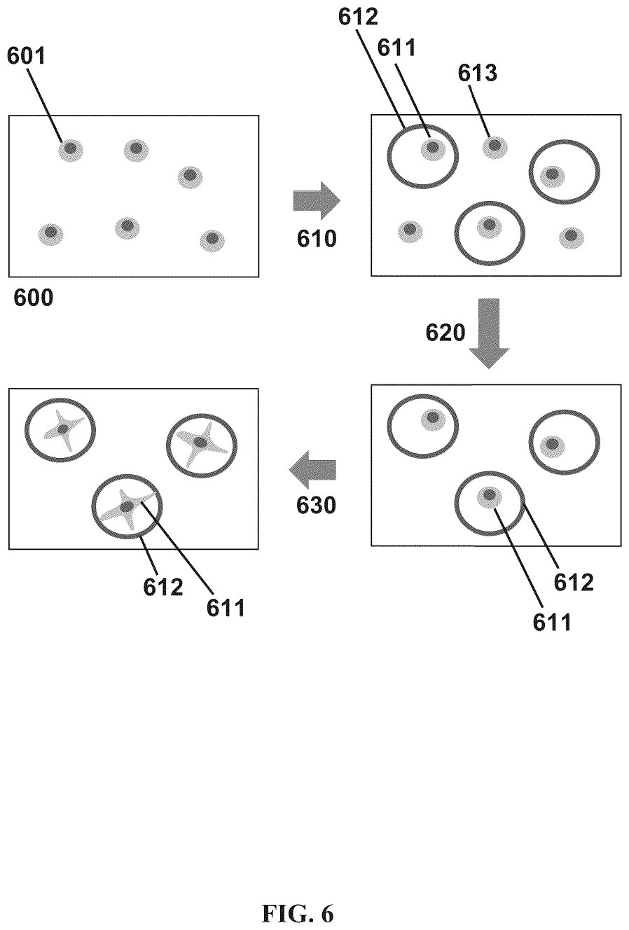

FIG. 6 illustrates an exemplary method of separating a plurality of cells. In an initial configuration (600), a channel of a fluidic device includes a plurality of cells (601). The plurality of cells (601) can be suspended, disposed on and/or adhered to a surface of the channel (or a species coupled thereto), or a combination thereof. In some cases, at least a subset of the plurality of cells (601) are adhered to the surface. However, as detailed further herein, in other cases the plurality of cells (601) are contacted to an adhesion inhibitor that limits or prevents adherence, or subsequent steps are performed before the plurality of cells (601) are able to adhere to the surface. The plurality of cells can be input into the channel, cultured (e.g., differentiated, proliferated, etc.) in the channel, or a combination thereof.

The method can include synthesizing (610) one or more chambers (612) that at enclose first cells (611) of the plurality of cells. The synthesizing can result in second cells (613) of the plurality of cells not being enclosed or partially enclosed within the one or more chambers (612). Such second cells (613) may be referred to as “interstitial cells” in reference to their positions outside of the one or more chambers. As detailed further herein, interstitial cells (613) can release soluble factors and deplete local nutrient and oxygen concentrations to influence the physiology and behavior of chamber-enclosed cells (611). Interstitial cells (613) may thus be removed (620) from the channel or, alternatively, moved to a separate portion of the channel such as a back end of the channel away from the one or more chambers. The interstitial cells (613) may be removed (620) immediately after chamber (612) synthesis (610) to prevent unintended early interactions or baseline contamination. Alternatively, the interstitial cells (613) may be removed (620) following an incubation period that may range from several minutes to multiple days depending on experimental design, environmental conditions, or the desired level of cellular interaction. The interstitial cells (613) may reside in the fluidic device for minutes, hours, days, or longer to allow the interstitial cells (613) to interact with the first cells (611) that are enclosed in chambers (612). During this incubation period, the interstitial cells (613) may interact with the first cells (611), for example by secreting soluble factors that diffuse into the chambers (612) and contact the first cells (611), by sequestering secretions from the first cells (611), by projecting growths (e.g., neurites or filopodia) through walls of the chambers (612) that contact the first cells (611), by contacting growths from the first cells (611) that project through walls of the chambers (612) and contact the interstitial cells (613), by depleting nutrients within the fluidic device, or a combination thereof. As examples, these interactions may include bidirectional chemical signaling, localized extracellular matrix remodeling, or mechanical feedback through the chamber boundaries that alter cell morphology or function. Additionally, gradients of oxygen, metabolites, or signaling molecules may develop over time, further influencing the behavior of both interstitial and enclosed cells. Interactions between the interstitial cells (613) and first cells (611) may be monitored continuously or periodically using one or more imaging and/or biochemical analysis methods disclosed herein. These methods can reveal both transient and sustained cellular responses, enabling researchers to map communication pathways and define states of the first (611) and interstitial cells (613) based on their interactions and local cell communities. The interstitial cells (613) may then be removed (620) from the fluidic device using one or more techniques disclosed herein.

Optionally, during an incubation (630), the enclosed cells (611) can adhere to surfaces of the one or more chambers, such as a top surface of the channel, a bottom surface of the channel, a wall of the channel, a surface of a chamber, another structure within the channel, or a combination thereof. For example, after an incubation step (630), the enclosed cells (611) can transition to an adherent state. Alternatively, the enclosed first cells (611) can also remain suspended within the one or more chambers or adhere to a surface prior to removal (620) of the interstitial cells (613).

For example, an aspect of the present disclosure provides a method for separating one or more cells that comprises inputting the plurality of cells into a fluidic device; synthesizing one or more chambers in the fluidic device to enclose a first cell of the plurality of cells and not enclose a second cell of the plurality of cells, and removing the second cell from the fluidic device; wherein the removing comprises contacting the second cell with a cell detachment reagent that detaches the second cell from a first surface of the fluidic device or from an adherent support coupled to the first surface of the fluidic device, and wherein the first cell remains enclosed in the one or more chambers during the removing (e.g., the one or more chambers remain intact and continue to encapsulate the first cell during and optionally following second cell removal). In a particular aspect, the present disclosure provides a method for separating one or more cells that comprises inputting the plurality of cells into a channel of a fluidic device; synthesizing one or more chambers in the channel to enclose a first cell of the plurality of cells and not enclose a second cell of the plurality of cells, and removing the second cell from the channel; wherein the removing comprises contacting the second cell with a cell detachment reagent that detaches the second cell from a first surface of the channel or from an adherent support coupled to the first surface of the channel, and wherein the first cell remains enclosed in the one or more chambers during the removing. As used herein, synthesizing one or more chambers to not enclose a second cell of a plurality of cells can denote that the second cell is not enclosed within a chamber of the one or more chambers. In select embodiments, synthesizing one or more chambers to not enclose a second cell of a plurality of cells denotes that the second cell is not trapped within a fluidic device or channel by the one or more chambers (e.g., surrounded by a collection of chambers of the one or more chambers in a manner that prevents the second cell from flowing out of the fluidic device or channel).

It is worthwhile to note that the first cell may be enclosed based on a phenotypic feature such as a cytokine secretion, fluorescent intensity and/or wavelength, shape of cell (e.g., area, spherical, ellipse shape, and/or perimeter), and combinations thereof. Similarly, the second cell may not be enclosed based on a phenotypic feature such as a cytokine secretion, fluorescent intensity and/or wavelength, shape of cell (e.g., area, spherical, ellipse shape, and/or perimeter), and combinations thereof. Such discrimination in cell disclosure can provide an advantage in analyzing a particular cell type that is disclosed and washing away other cell types that could potentially conflate the analysis. In another embodiment, the first cell and second cell can be of the same type, but the second cell is not enclosed due to geometric limitations where it is not possible to enclose a single cell due to the proximity of other adjacent cells or due to an undesirable overlap of the walls of adjacent enclosures.

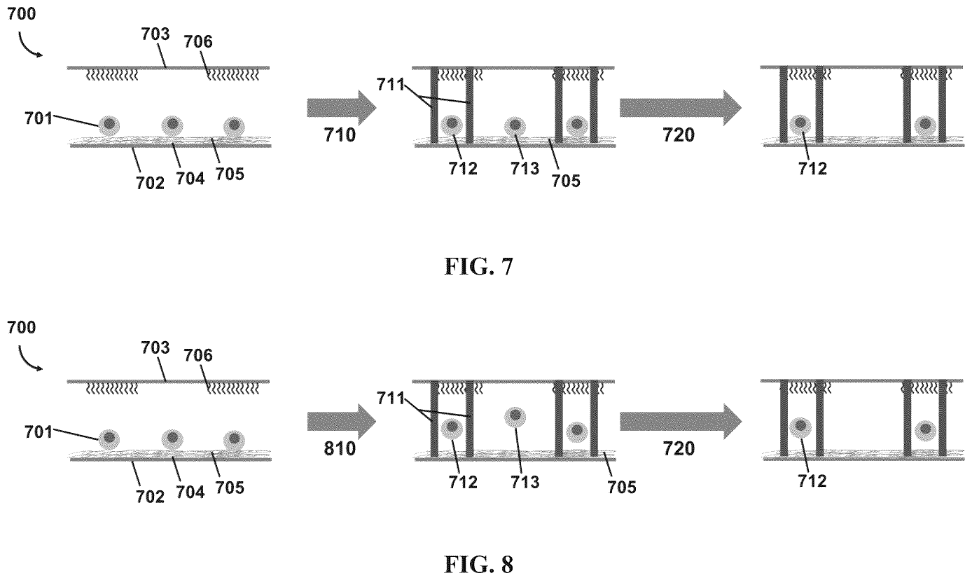

A version of this method is illustrated in FIG. 7, which depicts a fluidic device channel (700) that comprises a plurality of cells (701) disposed on a cell adherent support (705) on a bottom surface (702) of the channel (700). While in this illustration the cell adherent support (705) is coupled to an additional support (704) that is connected to the bottom surface (702) of the channel (700), in alternate embodiments, the cell adherent support (705) is directly attached to the bottom surface (702) or, along with the additional support (704), is absent, such that the plurality of cells (701) are exposed to the bottom surface (702). FIG. 7 further depicts capture elements (706) coupled to a top surface (703) of the channel (700) that may capture one or more species from the plurality of cells such as nucleic acids or proteins.

The method can include synthesizing (710) one or more chambers (711) that enclose first cells (712) of the plurality of cells within the channel and do not enclose second cells (713) of the plurality of cells. In FIG. 7, a second cell is depicted as being disposed on the cell adherent support (705). This depiction illustrates that the second cells (713) can be adhered to the cell adherent support (705), rendering the second cells resistant to removal through simple washing or agitation steps. Such “interstitial” cells (713) can affect the biochemistry and behavior of enclosed cells (712) in the fluidic channel, for example by releasing soluble factors into media surrounding an enclosed cell (712) or projecting through a pore of the one or more chambers (711) to directly contact the enclosed cell (712).

To address this problem, the present application provides methods (720) for removing adhered interstitial cells (713) from fluidic device channels (700), enabling enclosed cells (712) to be studied in controlled environments. As detailed further below, adhered interstitial cell removal (720) can include contacting such cells with cell detachment reagents such as EDTA or trypsin that dissociate the cells from fluidic device surfaces.

As used herein, a “cell adherent support” can denote a species that promotes cell binding or adherence. Many cells readily adhere to constituents of extracellular matrices (ECMs) such as particular glycosaminoglycans or fibronectin. Accordingly, a cell adherent support can be an ECM biomolecule. In some cases, the cell adherent support is selected from actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof. The cell adherent peptide can comprise a sequence recognized by one or more integrins, for example GFOGER (wherein ‘O’ denotes hydroxyproline) (SEQ ID NO: 1), YIGSR (SEQ ID NO: 2), LRE, GRGDS (SEQ ID NO: 3), CKKQRFRHRNRKG (SEQ ID NO: 4), KRSR (SEQ ID NO: 5), VPGIG (SEQ ID NO: 6), MNYYSNS (SEQ ID NO: 7), CSVTCG (SEQ ID NO: 8), GFRGDGQ (SEQ ID NO: 9), HAV, FLPASGL (SEQ ID NO: 10), or a combination thereof.

An “additional support” can be a substrate that binds to a fluidic device surface and a cell adherent support. Additional supports are often proteins or polymers. When the cell adherent support is charged, the additional support, when present, is often oppositely charged relative to the cell adherent support to promote binding. For example, when the cell adherent support is negatively charged, the additional support, when present, may be a polycation such as poly-l-ornithine, polylysine, or chitosan. When the cell adherent support is positively charged, the additional support, when present, may be a polyanion such as polyacrylate or polyaspartate.

In many cases, prior to its removal, the second cell is adhered to the first surface of the channel or to the cell adherent support coupled to the first surface of the channel. In such cases, contacting the second cell with the cell detachment reagent causes the second cell to separate from the first surface of the channel or from the cell adherent support coupled to the first surface of the channel, for example to become suspended within the fluidic channel and amenable to removal in a mild wash step.

Retaining target cells during nontarget cell removal is often a challenge in cell separation methods. In some embodiments, the present disclosure addresses this issue by enclosing target cells in custom-synthesized chambers that prevent their transport during wash, treatment, agitation, or other removal steps. Accordingly, a method for removing the second cell can cause the first cell to become suspended within the one or more chambers without causing it to be removed. The method can include incubating the first cell following the removing, during which incubating the first cell binds or adheres to the first surface of the channel or to the cell adherent support coupled to the first surface of the channel.

The one or more chambers can enclose a capture element with the first cell. The capture element can optionally be coupled to a top or bottom surface of the channel of the fluidic device. The capture element can alternatively be coupled to surface of a chamber or to a nano or microstructure such as a bead disposed within the channel. In some cases, the one or more chambers co-enclose the capture element and the cell adherent support with the first cell. In such designs, the cell adherent support can be coupled to the first surface of the channel and the capture element is coupled to a second surface of the channel opposite the first surface of the channel. Alternatively, the cell adherent support and the capture element can be coupled to the first surface of the channel. For example, the cell adherent support can be adjacent to the capture element on the first surface of the channel.

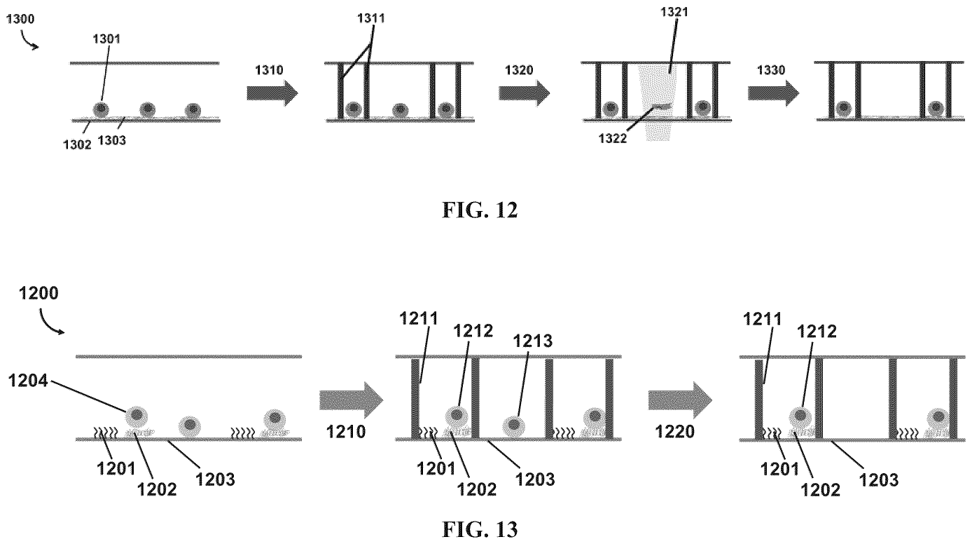

An example of a method that utilizes such a design is depicted in FIG. 12. In this method, the channel (1200) includes a surface (1203) with oligonucleotides (1201) adjacent to a cell adherent support (1202). A plurality of cells (1204) are input into the channel and a subset of the cells bind to the cell adherent support (1202). One or more chambers (1211) can be synthesized (1210) enclosing portions of the channel (1200) that include oligonucleotides (1201) and a cell adherent support (1202), thereby at least partially enclosing first cell of the plurality of cells that associates with or binds to the cell adherent support, and disposing the first cells in chambers with oligonucleotides (1201). Cells that are not adhered to the cell adherent support (1213) are not enclosed within the one or more chambers, and can be removed (1220) in subsequent wash steps.

Interstitial cell prevalence may also be limited by preventing cell adherence prior to chamber synthesis and non-chamber-enclosed cell removal. This method can include forcing cells to remain in suspension until interstitial cells are removed from the channel, and then allowing enclosed cells to adhere to channel surfaces. While simple wash steps can be insufficient for removing even poorly adhered interstitial cells, intensive detachment steps can alter the phenotype and viability of cells retained for further analysis (e.g., cells enclosed in a chamber). Many proteases that are used for cell surface detachment also target multiple surface markers that are not involved in cell attachment but whose cleavage can alter cell phenotype and behavior (Shahinian et al. Expert Review of Proteomics, 2013; 10(5):421). For example, trypsin has been shown to modulate CD44, CD55, CD73, CD105, CD140a, CD140b, and CD201 expression in mesenchymal stem cell populations (Tsuji et al. Cell Transplantation, 2017; 26:1089) and to potentiate differentiation in fibrocytes (White et al. PLoS ONE 8(8):e70795). Intensive wash steps can also strip or otherwise alter cell adherent coatings such as fibronectin and laminin on fluidic device surfaces, limiting assay repeatability and cell viability within a fluidic device. Accordingly, it can be advantageous to inhibit cell adherence to enable the use of low intensity cell removal methods.

Following from this discovery, in one aspect, the present disclosure provides a method for separating cells that includes inputting a plurality of cells into a fluidic device; contacting the plurality of cells with an adhesion inhibitor that does not prevent binding of a first cell of the plurality of cells to a surface of the fluidic device and diminishes a binding affinity of a second cell of the plurality of cells for the surface of the fluidic device; and removing the second cell from the fluidic device, wherein the first cell remains in the fluidic device following the removing. In a particular aspect, the present disclosure provides a method for separating cells that includes inputting a plurality of cells into a channel of a fluidic device; contacting the plurality of cells with an adhesion inhibitor that does not prevent binding of a first cell of the plurality of cells to a surface of the channel and diminishes a binding affinity of a second cell of the plurality of cells for the surface of the channel; and removing the second cell from the channel, wherein the first cell remains in the channel following the removing.

A method can also utilize one or more chambers to prevent target cell removal. For example, another aspect of the present disclosure provides a method for separating cells that includes inputting a plurality of cells into a fluidic device; contacting the plurality of cells with an adhesion inhibitor that diminishes a binding affinity of at least a subset of the plurality of cells to a first surface of the fluidic device or a cell adherent support coupled to the first surface of the fluidic device; synthesizing one or more chambers in the fluidic device to enclose first cells of the plurality of cells and not enclose second cell of the plurality of cells; and removing the second cell from the fluidic device, wherein the first cell remains enclosed in the one or more chambers during the removing. In a further aspect, the present disclosure provides a method for separating cells that includes inputting a plurality of cells into a channel of a fluidic device; contacting the plurality of cells with an adhesion inhibitor; synthesizing one or more chambers in the channel to enclose first cells of the plurality of cells and not enclose second cell of the plurality of cells; and removing the second cell from the channel, wherein the first cell remains enclosed in the one or more chambers during the removing. As detailed further herein, the adhesion inhibitor may diminish a binding affinity of at least a subset of the plurality of cells to a first surface of the channel or a cell adherent support coupled to the first surface of the channel, for example by blocking adhesion proteins on surfaces of the cells or sequestering metals that couple to cell surface proteins and promote surface adherence. The diminished binding affinity can be a diminished binding strength, for example wherein a cell adheres less strongly to a surface than it would in the absence of the adhesion inhibitor. The diminished binding affinity can also be a diminished binding rate, for example wherein a cell adheres to a surface more slowly than it would in the absence of the adhesion inhibitor. In certain cases, the diminished binding affinity may be a combination of diminished binding strength and diminished binding rate.

The removing can further comprise contacting the second cell with a cell detachment reagent that diminishes an affinity of the second cell for the first surface of the channel. In such cases, the removing can comprise a lower concentration of the cell detachment reagent or milder conditions than would be required in the absence of the inhibitor. Alternatively, the adhesion inhibitor can prevent the need for a cell detachment reagent, and the second cell can be removed from the fluidic device channel in a wash step.

An example of this method is shown in FIG. 8, which depicts a plurality of cells (701) in a fluidic cell channel (700) that includes a bottom surface (702) and top surface (703) along with optional substrates (704), cell adherent supports (705), and capture elements (706) as described for FIG. 7. In this example, the plurality of cells are contacted to an adhesion inhibitor (810) that limits cell binding to a surface (702, 703) or support (705) in the channel. While in FIG. 8 this step is depicted as occurring subsequently to cell loading into the channel (700), this step may be performed prior to or concurrently with cell loading and before or after chamber (711) synthesis. For example, the method can include a pre-incubation step in which at least a subset of the plurality of cells (701) are coupled to an adhesion inhibitor prior to loading into the channel (700).

First cells of the plurality of cells (712) can be enclosed within one or more chambers (711), leaving second cells of the plurality of cells (713) in interstitial spaces outside of the one or more chambers (711). While FIG. 8 depicts the enclosed (first, 712) and interstitial (second, 713) cells as being suspended, one or more of the cells can be disposed on a substrate (705) or surface (702, 703) of the channel (700), for example if the adhesion inhibitor weakens but does not prevent cell adhesion.

The interstitial cells (713) can be removed (720) from the channel (700). For cases in which the interstitial cells (713) are suspended, the cells may be removed with one or more low intensity wash steps that minimally perturb the enclosed cells (712). The wash steps can also remove adhesion inhibitor coupled to the enclosed cells (712) to promote adherence of the enclosed cells to a surface (702, 703) of the fluidic device channel (700). Alternatively, the enclosed cells (712) may remain suspended following interstitial cell removal (713).

The plurality of cells can be contacted to the adhesion inhibitor before they are input into the channel of the fluidic device. The adhesion inhibitor can bind to the plurality of cells or alter their affinity for one or more substrates. For example, the adhesion inhibitor can remove divalent cations from surface proteins expressed by the plurality of cells to diminish integrin binding by the plurality of cells. Accordingly, in such cases, the cells exhibit diminished binding to the surfaces of the channel.

Alternatively, the plurality of cells can be contacted to the adhesion inhibitor concurrently or subsequently to their input into the fluidic device channel. As some cells require multiple minutes or hours in quiescent conditions to bind to surfaces, these cells may be contacted to the adhesion inhibitor after they are distributed within the channel. The adhesion inhibitor may prevent the plurality of cells from binding to surfaces, or may limit further surface binding by the plurality of cells to render them easier to remove from the fluidic device. In some cases, the adhesion inhibitor is loaded into the channel with one or more polymer precursors of the one or more chambers.

As used herein, the term “adhesion inhibitor” refers to a species that diminishes cell binding affinity to a solid substrate such as poly-l-ornithine, glass, or laminin. As many cells utilize integrins to bind to surfaces, in many cases, the adhesion inhibitor diminishes an integrin binding affinity of the plurality of cells. Two primary means of diminishing integrin binding affinity are (1) contacting cells with an adhesion inhibitor that binds to integrins, thereby blocking the integrins from binding to fluidic device surfaces, and (2) contacting cells with an adhesion inhibitor that modulates integrin binding affinity, thereby lowering integrin affinity for fluidic device surfaces. Examples of species that block integrin binding include antibodies, collagen, gelatin, actinin, collagen, fibrinogen, fibronectin, ICAM-1, ICAM-2, laminin, osteopontin, paxillin, poly-l-lysine (PLL), poly-d-lysine (PDL), poly-l-ornithine, talin, VCAM-1, vinculin, vitronectin, and cell adherent peptides, as well as combinations thereof. However, the adhesion inhibitor may alternatively or additionally target a non-integrin protein involved in cell adherence, such as dystroglycan, 37/67LR, intercellular adhesion molecule (ICAM), or vascular adhesion molecule (VCAM); or a cell surface polysaccharide involved in cell adhesion such as a glycolipid or bacterial polysaccharide. In many cases, the adhesion inhibitor is dissolved within the channel of the fluidic device. For example, when the adhesion inhibitor comprises fibronectin, the fibronectin can be introduced into the channel in a solubilized, non-aggregated or partially aggregated form.

A cell adherent peptide can comprise a linear or cyclic peptide with about 3 to 200 amino acids, about 3 to 100 amino acids, about 4 to 50 amino acids, about 4 to 25 amino acids, about 4 to 15 amino acids, or about 4 to 10 amino acids that comprises one or more integrin-binding sequences, which are typically derived from integrin substrates such as fibronectin, elastin, and laminin. Non-limiting examples of integrin-binding sequences are listed in Table 1. Further examples of integrin-binding sequences are listed in U.S. Patent Publication 20210009763A1; U.S. Pat. No. 8,981,046B2; Ludwig et al., Cancers (Basel), 2021; 13(7):1711; Schaffner P & Dard, Cell MoLife Sci., 2003, 60(1):119-32; and Hersel et al., Biomaterials, 2003, 24(24):4385-415, which are incorporated herein by reference. A cell adherent peptide may contain one or more of the sequences listed in Table 1 or in U.S. Patent Publication 20210009763A1, U.S. Pat. No. 8,981,046B2, Ludwig et al., Schaffner P & Dard, or Hersel et al. For example, a peptide may comprise the sequence RGD (-arginine-glycine-aspartic acid-) flanked by zero to six leading amino acids and zero to six tailing amino acids. Small molecule mimetics of cell adherent peptides, for example those disclosed in Slack et al., Nat. Rev., 2022; 21:60-78 and Pang et al., Signal Transduction and Targeted Therapy, 2023; 8:1 which are herein incorporated by reference, may be used in place of or in combination with cell adherent peptides.

TABLE 1

| SEQ ID NO | Sequence | Source Protein(s) |
|---|---|---|
| — | RGD | Fibronectin, Vitronectin, von Willebrand |
| | | Factor, Fibrinogen, Osteopontin, |
| | | Thrombospondin, Latency-Associated Protein |
| SEQ ID NO: 1 | GFOGER | Collagen 1 |
| SEQ ID NO: 2 | YIGSR | Laminin |
| — | LRE | Laminin Subunit γ1 |
| SEQ ID NO: 3 | GRGDS | Fibronectin |
| SEQ ID NO: 4 | CKKQRFRHRNRKG | Vitronectin |
| SEQ ID NO: 5 | KRSR | Osteopontin |
| SEQ ID NO: 6 | VPGIG | Elastin |
| SEQ ID NO: 7 | MNYYSNS | Collagen 4 |
| SEQ ID NO: 8 | CSVTCG | Thrombospondin |
| SEQ ID NO: 9 | GFRGDGQ | Nidogen-1 |
| — | HAV | N-Cadherin |
| SEQ ID NO: 10 | FLPASGL | TGF-β1 |

An adhesion inhibitor can also include an antibody that binds to a cell surface marker involved in adhesion. In many cases, the antibody is an integrin-binding antibody. Large numbers of integrin-binding antibodies are known. Table 2 lists exemplary references that disclose antibodies and which are incorporated herein by reference.

TABLE 2

| Reference | Integrin Antibodies |
|---|---|
| U.S. Patent 11,267,891B2 | Pan-α2 antibodies |
| Ley et al., Nat Rev Drug Discov., 2016; | Anti-αIIbβ3, α4β1, α4β7, αLβ2, α4β1, αEβ7 |
| 15(3): 173-183. | antibodies |
| Byron et al., J Cell Sci., 2009; 122(22): | Pan-α1, α2, α3, α4, α5, α6, α7, α9, αV, αE, αL, αM, |
| 4009-4011. | αX, αD, β1, β2, β3, β4, β5, β6, β7, β8, and anti- |
| | αIIbβ3, αVβ3, αVβ5, αVβ6, and αLβ2 antibodies |
| Slack et al., Nat. Rev., 2022; 21: 60-78. | Pan-α4, αL, αv, β1, β2, β3, β7 and anti-αLβ2, α4β1, |
| | α4β7, α5β1, αLβ2, αvβ1, αvβ3, αvβ5, αvβ6, αvβ8, |
| | αIIbβ3 antibodies |
| Pang et al., Signal Transduction and | Pan-αv, α2, α4, α6, β1, and anti-α1β1, α2β1, α4β1, |
| Targeted Therapy, 2023; 8:1. | α4β7, α5β1, α9β1, αvβ1, αvβ3, αvβ5, αvβ6, αIIbβ3 |
| | antibodies |

Examples of species that modulate integrin binding affinity include metal chelators, and in particular divalent cation chelators such as EDTA. In many cases, the metal chelator includes a formation constant (K_{f}), defined herein according to Scheme 1 and Equation 1 below, wherein Mⁿ⁺ is a metal ion with charge n+, Y^{m-} is a metal chelator with charge m−, and MY^{n-m} is complex of the metal ion and metal chelator with charge n−m. As K_{f} is a ratio of complex to free metal ion and free metal chelator concentrations, higher K_{f} values indicate higher metal ion affinities for a particular metal chelator. In general, metal chelators disclosed herein include formation constants for Ca²⁺, Mg²⁺, and Mn²⁺, three divalent ions that modulate integrin binding affinity, of at least about 10⁶, about 10⁷, about 10⁸, about 10⁹, or about 10¹⁰ at 20° C. and ionic strength of 0.1 M.

$${M}^{n +} + {Y}^{m -} ⇌ {MY}^{n - m} \qquad ( SCHEME 1 )$$

$${K}_{f} = \frac{[ M {Y}^{n - m} ]}{[ {M}^{n +} ] [ {Y}^{m -} ]} \qquad ( EQUATION 1 )$$

Examples of metal chelators consistent with the present disclosure include ethylenediaminetetraacetic acid (EDTA), glutamic acid diacetate (GLDA), dicarboxymethyl alaninate, glutamate diacetate, nitrilotriacetic acid (NTA), 1,4,7,10-tetraazacyclododecane-1,4,7,10-tetraacetic acid, (DOTA), diethylenetriaminepentaacetic acid (DTPA), 1,4-bis(carboxymethyl)-6-[bis(carboxymethyl)]amino-6-methylperhydro-1,4-diazepine (AAZTA), 2,2′-(6-((carboxymethyl)amino)-1,4-diazepane-1,4-diyl)diacetic acid (DATA), N,N′-bis(2-hydroxybenzyl)-ethylenediamine-N,N′-diacetic acid, (HBED), bis(2-pyridylcarbonyl) amine (BPCA), 4-acetylamino-4-[2-[(3-hydroxy-1,6-dimethyl-4-oxo-1,4-dihydro-pyridin-2-ylmethyl)-carbamoyl]-ethyl]-heptanedioicacid bis-[(3-hydroxy-1,6-dimethyl-4-oxo-1,4-dihydro-pyridin-2-ylmethyl)-amide](CP256), desferrioxamine B (DFO), 1,4,7,10,13,16-hexaazacyclohexadecane-N,N′,N″,N′″,N″″,N′″″-hexaacetic acid (HEHA), {4-[2-(bis-carboxymethylamino)-ethyl]-7-carboxymethyl-[1,4,7]triazonan-1-yl}-acetic acid (NETA), 1,4,7-triazacyclononane-1,4-bis[methylene(hydroxymethyl)phosphinic acid]-7-[methylene(2-carboxyethyl)phosphinic acid](NOPO), 1,4,7-triazacyclononane-1,4,7-triacetic acid, (NOTA), 3,6,9,15-tetraazabicyclo[9.3.1]-pentadeca-1(15),11,13-triene-3,6,9,-triacetic acid (PCTA), 1,4,7,10,13-pentaazacyclopentadecane-N,N′,N″,N′″,N″″-pentaacetic acid (PEPA), N,N′-bis(2-hydroxy-5-sulfobenzyl)-ethylenediamine-N,N′-diacetic acid (SHBED), N,N′,N″, tris(2-mercaptoethyl)-1,4,7-triazacyclononane (TACN-TM), 1,4,8,11-tetraazacyclotetradecane-1,4,8,11-tetraacetic acid, (TETA), and 1,4,7-triazacyclononane-1,4,7-tris[methyl(2-carboxyethyl)phosphinic acid](TRAP).

Adhesion inhibitors, and in particular species that block integrin binding, can be released from the first cell. For example, a species that blocks integrin binding such as an RGD peptide may remain bound to the first cell following removal of the second cell from the fluidic device channel, which may diminish its affinity for a surface of the channel. The first cell, enclosed within one or more chambers and therefore retained during wash steps, may then be subjected to further wash, chemical treatment, and/or physical treatment steps to remove the species that blocks integrin binding. For example, the first cell may be contacted with a cell detachment reagent, heated, sonicated, and/or incubated in a divalent cation-free solution to release one or more species that block integrin binding. Similarly, divalent cations, and in particular Ca²⁺, Mg²⁺, and/or Mn²⁺ may be reintroduced to the first cell following second cell removal to enhance or modulate integrin binding. The first cell may thus be induced to adhere to a surface of the fluidic device following removal of the second cell.

In certain methods disclosed herein, one or more cells bind to oligonucleotide barcodes (e.g., nucleic acid capture probes) disposed on a channel surface or to cell adherent supports coupled to the oligonucleotide barcodes. Such a method can include inputting the plurality of cells into a fluidic device (e.g., a channel of a fluidic device) comprising a first surface comprising a plurality of oligonucleotides coupled to the first surface, wherein a first cell of the plurality of cells associates with a first subset of oligonucleotides of the plurality of oligonucleotides; synthesizing one or more chambers in the fluidic device to co-enclose the first cell with the first subset of oligonucleotides and not enclose a second cell of the plurality of cells, and removing the second cell from the fluidic device, wherein the first cell remains enclosed in the one or more chambers during the removing. In a particular aspect, the method includes inputting the plurality of cells into a channel of a fluidic device comprising a first surface comprising a plurality of oligonucleotides coupled to the first surface, wherein a first cell of the plurality of cells associates with a first subset of oligonucleotides of the plurality of oligonucleotides; synthesizing one or more chambers in the channel to co-enclose the first cell with the first subset of oligonucleotides and not enclose a second cell of the plurality of cells, and removing the second cell from the channel, wherein the first cell remains enclosed in the one or more chambers during the removing. In many cases, the second cell does not associate with oligonucleotides of the plurality of oligonucleotides or bind to the cell adherent supports coupled to the oligonucleotides of the plurality of oligonucleotides. Instead, the second cell can be adhered to the first surface or to an additional cell adherent support coupled to the first surface prior to the removing, which can comprise detaching the second cell from the first surface or from the additional cell adherent support coupled to the first surface. Alternatively, the second cell can be suspended within the channel. For example, the plurality of cells may bind to the first surface or to the additional cell adherent support coupled to the first surface 10, 50, 100, 500, or 1000 or more times more slowly than they associate with the oligonucleotides or bind to the cell adherent support optionally coupled to the oligonucleotides.

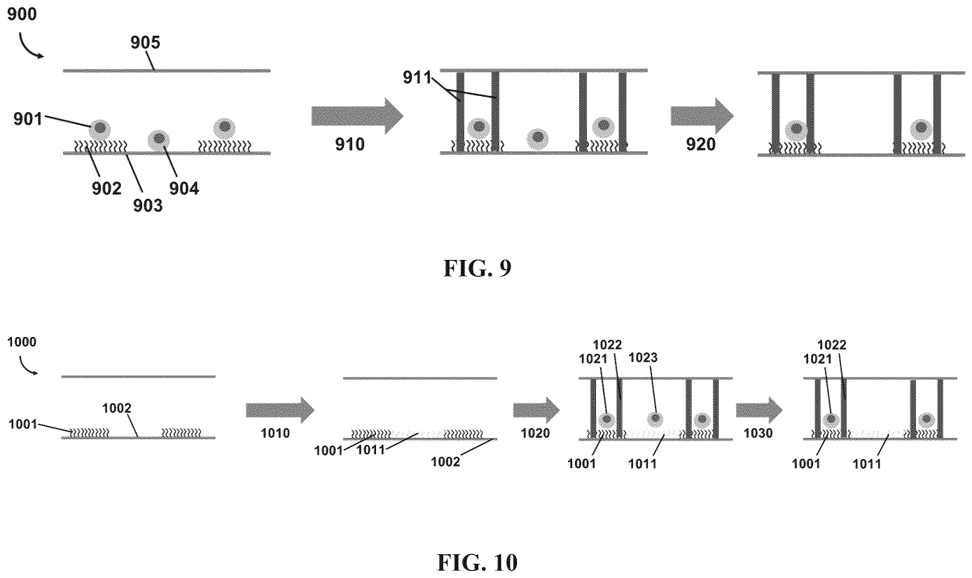

In some cases, the first cell associates with the oligonucleotides of the plurality of oligonucleotides. For example, the first cell may intercalate into or partition onto the oligonucleotides of the plurality of nucleotides. Optionally, the first cell may bind to the oligonucleotides. An example of this method is depicted in FIG. 9, which illustrates a fluidic device channel (900) comprising a plurality of cells of which a first cell (901) associates with an oligonucleotide (902) coupled to a first surface (903) of a channel and a second cell (904) couples to a first surface (903), another substrate (905) or remains suspended within the channel (900). One or more chambers (911) can then be synthesized (910) that at least partially enclose the first cell (901) and do not enclose the second cell (904). The second cell (904) can then be removed (920) from the channel (900), during which removal the first cell (901) remains confined within the one or more chambers (911).

Alternatively, the first cell may bind to cell adherent supports coupled to the oligonucleotides of the plurality of oligonucleotides. For example, a method for separating a plurality of cells can comprise inputting the plurality of cells into a channel of a fluidic device comprising a first surface comprising a plurality of oligonucleotides coupled to the first surface and a cell adherent support coupled to the plurality of oligonucleotides, wherein a first cell of the plurality of cells binds to the cell adherent support; synthesizing one or more chambers in the channel to co-enclose the first cell with a first subset of oligonucleotides of the plurality of oligonucleotides and not enclose a second cell of the plurality of cells, and removing the second cell from the channel, wherein the removing comprises contacting the second cell with a cell detachment reagent, and wherein the first cell remains enclosed in the one or more chambers during the removing. In some cases, prior to the removing, the second cell is associated with a second subset of the plurality of oligonucleotides or is bound to the cell adherent support coupled to the second subset of the plurality of oligonucleotides. Alternatively, in some cases, the second cell does not bind to the cell adherent support coupled to the plurality of oligonucleotides.

In certain methods disclosed herein, oligonucleotide barcodes (e.g., nucleic acid capture probes), cell adherent supports coupled to oligonucleotide barcodes, or cell adherent supports coupled to a surface of a fluidic device may be selectively removed from a channel surface to facilitate interstitial cell detachment and removal. In some cases, barcode and/or cell adherent supports are removable with light. The light may be selectively applied to interstitial regions with a spatial light modulator (SLM) such as a digital micromirror device (DMD). The oligonucleotide barcodes and/or cell adherent supports can include one or more photocleavable groups such as an arylcarbonylmethyl group, an o-alkylphenacyl group, a p-hydroxyphenacyl group, a benzoin group, a nitroaryl group, a nitroanilide group, or a coumarin-4-ylmethyl group.

Alternatively, a reagent that cleaves oligonucleotidebarcodes, cell adherent supports coupled to the oligonucleotide barcodes, or cell adherent supports coupled to a surface of a fluidic device may be input into a fluidic device. The reagent may be prohibitively large to enter a chamber, and may thereby only remove species from interstitial spaces. For example, the reagent may be a PEGylated, XTENylated, or otherwise hydrodynamically enlarged enzyme that is incapable of diffusing through pores in walls of one or more chambers in a fluidic device.

As an example, a method for separating a plurality of cells can include inputting the plurality of cells into a channel of a fluidic device comprising a first surface comprising a plurality of oligonucleotides coupled to the first surface and a cell adherent support coupled to the plurality of oligonucleotides, wherein a first cell of the plurality of cells binds to the cell adherent support of a first subset of the plurality of oligonucleotides; synthesizing one or more chambers in the channel to co-enclose the first cell with the first subset of the oligonucleotides of the plurality of oligonucleotides and not enclose a second cell of the plurality of cells that is bound to the cell adherent support of a second subset of the plurality of oligonucleotides; and removing the second cell from the channel, wherein the removing comprises: cleaving the second subset of the oligonucleotides from the first surface, cleaving the cell adherent support from the second subset of the plurality of oligonucleotides, or cleaving the second subset of the plurality of oligonucleotides from the first surface and cleaving the cell adherent support from the second subset of the plurality of oligonucleotides; and wherein the first cell remains enclosed in the one or more chambers during the removing. Numerous cleavage methods are compatible with this method, including photocleavage (e.g., ultraviolet light-mediated cleavage), chemical cleavage, thermal cleavage, pH-mediated cleavage, and enzymatic (e.g., nuclease-mediated) cleavage. The second cell may be adhered to the first surface or to an additional cell adherent support coupled to the first surface prior to the removing. In such cases, the removing comprises detaching the second cell from the first surface or from the additional cell adherent support coupled to the first surface of the fluidic device.

Cell surface binding can also be mediated by coating portions of fluidic device surfaces with non-adherent supports. As used herein, the term “non-adherent support” can denote a species to which cells do not bind, or to which cells have a lowered binding affinity relative to the surface onto which they are coated. For example, a glass surface of a channel can be coated with a hydrophobic polymer such as polyethylene that blocks cell binding to the surface. In many cases, the non-adherent support comprises a synthetic polymer. Examples of non-adherent supports include acrylates, acrylamides, amines, silanes, PEG, PEG-silanes, acrylate-PEG-silanes, silane-PEG-silanes, PEG-amines, acrylamide-PEG-amines, acrylate-PEG-amines, COOH-PEG-amines, and combinations thereof.

In one aspect, the present disclosure provides a method for separating cells that comprises inputting the plurality of cells into a fluidic device, wherein the fluidic device comprises a first surface comprising: (a) a cell adherent support, and (b) a non-adherent support to which the plurality of cells comprise a diminished binding affinity relative to the cell adherent support; and performing a wash step that retains a first cell that is bound to the cell adherent support and removes a second cell that is disposed above, below, or on the non-adherent support. In a further aspect, the present disclosure provides a method for separating cells that comprises inputting the plurality of cells into a channel of a fluidic device, wherein the channel comprises a first surface comprising: (a) a cell adherent support, and (b) a non-adherent support to which the plurality of cells comprise a diminished binding affinity relative to the cell adherent support; and performing a wash step that retains a first cell that is bound to the cell adherent support and removes a second cell that is disposed above, below, or on the non-adherent support.

Such a method may include enclosing select cells within one or more chambers. For example, in particular aspects, the present disclosure provides a method for separating cells that comprises inputting the plurality of cells into a channel of a fluidic device, wherein the channel comprises a first surface comprising: (a) a cell adherent support, and (b) a non-adherent support to which the plurality of cells comprise a diminished binding affinity relative to the cell adherent support; synthesizing one or more chambers in the channel to co-enclose a first cell of the plurality of cells with the cell adherent support and not enclose a second cell of the plurality of cells that is disposed above, below, or on the non-adherent support; and removing the second cell from the channel; wherein the first cell remains enclosed in the one or more chambers during the removing.

An exemplary method for preparing and using a non-adherent support-coated channel is provided in FIG. 10. In this example, a fluidic device channel (1000) with a plurality of oligonucleotides (1001) coupled to a first surface (1002) is further functionalized (1010) with a non-adherent support (1011). The non-adherent support (1011) is deposited along portions of the first surface (1002) that do not contain the oligonucleotides (1001). This can be accomplished, for example, by spotting the oligonucleotides (1001) on regions of the first surface (1002) that are functionalized with reactive chemical handles, such as activated esters (e.g., NHS esters) or alkynes (e.g., for use in click chemistry coupling). The oligonucleotides (1001) may exhaust reactive chemical handles in the regions on which they are spotted, such that the non-adherent support (1011) may be flowed through the channel (1000) to react with portions of the surface (1002) not covered by oligonucleotides (1001). Alternatively, the method may include spotting the oligonucleotides (1001) and non-adherent support (1011) onto separate portions of the surface (1002). The method may also include spotting the non-adherent support (1011) onto regions of the surface (1002), and then flowing oligonucleotides (1001) through the channel (1000) to couple to unfunctionalized portions of the surface (1002). In some embodiments, multiple surfaces of the channel (1000) are functionalized with oligonucleotides (1001), non-adherent support (1011), or other species such as a cell adherent support (705) as depicted in FIGS. 7-8.

While oligonucleotides are depicted in the channel (1000) of FIG. 10, in alternate embodiments the oligonucleotides (1001) are replaced, applied concurrently with, or coupled to a cell adherent support (1011). For example, a cell adherent support can be coupled to the oligonucleotides (1001), disposed on a portion of the surface (1002) that contains the oligonucleotides (1001), or disposed in a separate portion of the channel (1000), such as a portion of a second surface opposite the oligonucleotides (1001).

A plurality of cells (1021, 1023) may be input into the channel (1000). Chambers (1022) may be synthesized (1020) around (i.e., enclosing) first cells (1021) of the plurality of cells that are disposed on or adjacent to the oligonucleotides (1001), wherein at least a portion of second cells (1023) that are disposed on or adjacent to the non-adherent support (1011) are not enclosed by the chambers (1022). The non-adherent support (1011) may limit cell binding to surfaces of the channel (1000), enabling their removal (1030) with mild wash steps.

The cell adherent support and non-adherent support can be patterned over at least a portion of the first surface. The channel can comprise a first plurality of regions comprising the cell adherent support and a second plurality of regions comprising the non-adherent support. The first and second pluralities of regions can be patterned along the surface. The pattern may be continuous (e.g., a checkerboard pattern comprised of alternating square-shaped regions that contain either the cell adherent support and the non-adherent support) or discontinuous (e.g., alternating rows of circular regions that contain either the cell adherent support or the non-adherent support and are interspersed by non-functionalized regions) along the first surface.

In many embodiments, the non-adherent support is disposed outside of the one or more chambers. In particular, the one or more chambers can be synthesized only in regions of the fluidic channel that contain the cell adherent support. Similarly, the one or more chambers can be synthesized only in regions of the fluidic channel that do not contain the non-adherent support. While in general the second cell does not bind to the non-adherent support, the second cell may weakly bind, intercalate within, or partition onto the non-adherent support. In such cases, removing the second cell from the fluidic device can comprise detaching the second cell from the non-adherent support, for example by inputting a cell detachment reagent into the fluidic device.

Numerous cell removal techniques are consistent with the presently disclosed methods. These methods enable facile interstitial cell removal, for example with relatively mild wash steps. In some cases, removing the second cell includes a single wash step or a plurality of wash steps. In some cases, removing the second cell comprises heating the channel of the fluidic device to between about 30-42° C. or about 30-37° C. In particular, the removing can comprise heating the channel from a chilled temperature (e.g., about 2° C., 5° C., 10° C. or 15° C.), for example to just below room temperature, to room temperature, or to about 30-42° C. or about 30-37° C. In some cases, removing the second cell comprises contacting the second cell with a cell detachment reagent such as EDTA or trypsin. Such a step can include inputting the cell detachment reagent into the channel of the fluidic device before, during, or after a wash step.

A wash solution can also include a low concentration of divalent ions, and in particular divalent ions such as Ca²⁺, Mg²⁺, and Mn²⁺ that modulate integrin binding. In some cases, the wash solution comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM Ca²⁺, less than about 10⁻⁴ mM, less than about 10⁻⁵ mM, or less than about 10⁻⁶ mM Ca²⁺. In some cases, the wash solution comprises between about 1 mM and 10⁻⁵ mM, between about 0.5 mM and 10⁻⁵ mM, between about 0.25 mM and 10⁻⁵ mM, between about 0.1 mM and 10⁻⁵ mM, between about 0.05 mM and 10⁻⁵ mM, between about 0.025 mM and 10⁻⁵ mM, between about 0.01 mM and 10⁻⁵ mM, between about 0.005 mM and 10⁻⁵ mM, between about 0.0025 mM and 10⁻⁵ mM, between about 0.001 mM and 10⁻⁵ mM, between about 10⁻⁴ mM and 10⁻⁵ mM, or between about 10⁻⁵ mM and 10⁻⁶ mM Ca²⁺. In some cases, the wash solution is essentially free of Ca²⁺ (i.e., does not contain an amount of Ca²⁺ that is detectable by atomic emission spectroscopy). In some cases, the wash solution comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM Mg²⁺, less than about 10⁻⁴ mM, less than about 10⁻⁵ mM, or less than about 10⁻⁶ mM Mg²⁺. In some cases, the wash solution comprises between about 1 mM and 10⁻⁵ mM, between about 0.5 mM and 10⁻⁵ mM, between about 0.25 mM and 10⁻⁵ mM, between about 0.1 mM and 10⁻⁵ mM, between about 0.05 mM and 10⁻⁵ mM, between about 0.025 mM and 10⁻⁵ mM, between about 0.01 mM and 10⁻⁵ mM, between about 0.005 mM and 10⁻⁵ mM, between about 0.0025 mM and 10⁻⁵ mM, between about 0.001 mM and 10⁻⁵ mM, between about 10⁻⁴ mM and 10⁻⁵ mM, or between about 10⁻⁵ mM and 10⁻⁶ mM Mg²⁺. In some cases, the wash solution is essentially free of Mg²⁺. In some cases, the wash solution comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM Mn²⁺, less than about 10⁻⁴ mM, less than about 10⁻⁵ mM, or less than about 10⁻⁶ mM Mn²⁺. In some cases, the wash solution comprises between about 1 mM and 10⁻⁵ mM, between about 0.5 mM and 10⁻⁵ mM, between about 0.25 mM and 10⁻⁵ mM, between about 0.1 mM and 10⁻⁵ mM, between about 0.05 mM and 10⁻⁵ mM, between about 0.025 mM and 10⁻⁵ mM, between about 0.01 mM and 10⁻⁵ mM, between about 0.005 mM and 10⁻⁵ mM, between about 0.0025 mM and 10⁻⁵ mM, between about 0.001 mM and 10⁻⁵ mM, between about 10⁻⁴ mM and 10⁻⁵ mM, or between about 10⁻⁵ mM and 10⁻⁶ mM Mn²⁺. In some cases, the wash solution is essentially free of Mn²⁺. In some cases, the wash solution comprises less than about 1 mM, less than about 0.5 mM, less than about 0.25 mM, less than about 0.1 mM, less than about 0.05 mM, less than about 0.025 mM, less than about 0.01 mM, less than about 0.005 mM, less than about 0.0025 mM, less than about 0.001 mM, less than about 10⁻⁴ mM, less than about 10⁻⁵ mM, or less than about 10⁻⁶ mM total divalent cations. In some cases, the wash solution comprises between about 1 mM and 10⁻⁵ mM, between about 0.5 mM and 10⁻⁵ mM, between about 0.25 mM and 10⁻⁵ mM, between about 0.1 mM and 10⁻⁵ mM, between about 0.05 mM and 10⁻⁵ mM, between about 0.025 mM and 10⁻⁵ mM, between about 0.01 mM and 10⁻⁵ mM, between about 0.005 mM and 10⁻⁵ mM, between about 0.0025 mM and 10⁻⁵ mM, between about 0.001 mM and 10⁻⁵ mM, between about 10⁻⁴ mM and 10⁻⁵ mM, or between about 10⁻⁵ mM and 10⁻⁶ mM total divalent cations. In some cases, the wash solution is essentially free of divalent cations.

In some cases, a wash step includes sonication, which herein denotes mechanical perturbations such as sound that modulate at one or more frequencies. In the disclosed methods, wash step sonication is typically sufficiently strong to detach cells from cell adherent supports but of insufficient power and duration to kill cells. In many cases, sonication is performed in combination with one or more additional methods for detaching cells from cell adherent supports, such as EDTA or trypsinization. The sonicating can include a frequency of about 0.02 to 0.25, about 0.02 to 0.5, about 0.02 to 1, about 0.02 to 10, about 0.02 to 25, about 0.02 to 50, about 0.02 to 100, about 0.02 to 250, about 0.02 to 500, about 0.02 to 1000, about 0.25 to 1, about 0.25 to 2.5, about 0.25 to 5, about 0.25 to 10, about 0.25 to 25, about 0.25 to 50, about 0.25 to 100, about 0.25 to 250, about 0.25 to 500, about 0.25 to 1000, about 1 to 10, about 1 to 25, about 1 to 50, about 1 to 100, about 1 to 200, about 1 to 500, about 1 to 1000, about 5 to 25, about 5 to 50, about 5 to 100, about 5 to 200, about 5 to 500, about 5 to 1000, about 10 to 25, about 10 to 50, about 10 to 100, about 10 to 200, about 10 to 500, about 10 to 1000, about 25 to 50, about 25 to 100, about 25 to 200, about 25 to 500, about 25 to 1000, about 50 to 100, about 50 to 200, about 50 to 500, about 50 to 1000, about 100 to 200, about 100 to 500, or about 100 to 1000 kilohertz (kHz). In some cases, the sonicating is ultrasonicating, which hereinafter denotes sonication with a frequency of at least about 16 kHz. In many cases, the sonication includes a duration of about 0.25 to 1, about 0.25 to 2.5, about 0.25 to 5, about 0.25 to 10, about 0.25 to 20, about 0.25 to 30, about 1 to 2.5, about 1 to 5, about 1 to 10, about 1 to 20, about 1 to 30, about 2.5 to 5, about 2.5 to 10, about 2.5 to 20, about 2.5 to 30, about 5 to 10, about 5 to 20, about 5 to 30, about 10 to 20, about 10 to 30, or about 20 to 30 minutes. The sonicating can include an intensity of about 0.25 to 1, about 0.25 to 2.5, about 0.25 to 5, about 0.25 to 10, about 0.25 to 25, about 0.25 to 50, about 1 to 2.5, about 1 to 5, about 1 to 10, about 1 to 25, about 1 to 50, about 2.5 to 5, about 2.5 to 10, about 2.5 to 25, about 2.5 to 50, about 5 to 10, about 5 to 25, about 5 to 50, about 10 to 50, or about 25 to 50 W/cm².

An interstitial cell removal step can also include the use of a cell detachment reagent. As used herein, a cell detachment reagent can be a species that diminishes cell adherence, for example by cleaving, blocking, or allosterically modulating cell surface proteins responsible for cell adherence. A cell detachment reagent can also alter conditions to disfavor cell adhesion, for example by sequestering divalent metal ions that bind to and modulate integrin binding affinities.

In certain cases, a cell detachment reagent comprises a protease, a metal chelator, a polyanion, or a combination thereof. In some cases, a cell detachment reagent comprises a protease, a metal chelator, or a combination thereof. The protease can be, for example, accutase, dispase, chymotrypsin, collagenase, GluC endoprotease, pepsin, trypsin, TrypLE, or a combination thereof. The protease can also be accutase, dispase, chymotrypsin, collagenase, GluC endoprotease, pepsin, trypsin, or a combination thereof. The protease can be present (e.g., in a wash solution) at about 0.05% to 0.1%, about 0.05% to 0.25%, about 0.05% to 0.5%, about 0.05% to 1%, about 0.05% to 1.5%, about 0.05% to 2%, about 0.05% to 2.5%, about 0.05% to 3%, about 0.05% to 4%, about 0.1% to 0.25%, about 0.1% to 0.5%, about 0.1% to 1%, about 0.1% to 1.5%, about 0.1% to 2%, about 0.1% to 2.5%, about 0.1% to 3%, about 0.1% to 4%, about 0.25% to 0.5%, about 0.25% to 1%, about 0.25% to 1.5%, about 0.25% to 2%, about 0.25% to 2.5%, about 0.25% to 3%, about 0.25% to 4%, about 0.5% to 1%, about 0.5% to 1.5%, about 0.5% to 2%, about 0.5% to 2.5%, about 0.5% to 3%, about 0.5% to 4%, about 1% to 1.5%, about 1% to 2%, about 1% to 2.5%, about 1% to 3%, about 1% to 4%, about 1.5% to 2%, about 1.5% to 2.5%, about 1.5% to 3%, about 1.5% to 4%, about 2% to 2.5%, about 2% to 3%, about 2% to 4% weight/volume (w/v) or weight/weight (w/w). The metal chelator can be, for example, EDTA, GLDA, dicarboxymethyl alaninate, NTA, DOTA, DTPA, AAZTA, DATA, HBED, CP256, DFO, HEHA, NETA, NOPO, NOTA, PCTA, PEPA, TACN-TM, TETA, TRAP, or a combination thereof. The metal chelator may be present at about 0.1 to 0.25, about 0.1 to 0.5, about 0.1 to 1, about 0.1 to 1.5, about 0.1 to 2.5, about 0.1 to 5, about 0.1 to 10, about 0.1 to 20, about 0.25 to 0.5, about 0.25 to 1, about 0.25 to 1.5, about 0.25 to 2.5, about 0.25 to 5, about 0.25 to 10, about 0.25 to 20, about 0.5 to 1, about 0.5 to 1.5, about 0.5 to 2.5, about 0.5 to 5, about 0.5 to 10, about 0.5 to 20, about 1 to 2.5, about 1 to 5, about 1 to 10, about 1 to 20, about 2.5 to 5, about 2.5 to 10, about 2.5 to 20, about 5 to 10, about 5 to 20, or about 10 to 20 mM. In particular cases, the metal chelator is a polyanion such as polyacrylic acid or alginate, and comprises a molecular weight of at least about 1 megadalton (MDa). In some cases, a metal chelator and protease are contacted to the cells. In such cases, the metal chelator may be contacted to the cells before the protease is contacted to the cells, the metal chelator may be contacted to the cells after the protease is contacted to the cells, the metal chelator and protease may be contacted to the cells simultaneously (e.g., within a single wash solution), or a combination thereof. In many cases, a method of removing causes the first cell and the second cell to become suspended within the fluidic device channel. As non-limiting examples, the polyanion can include polyacrylic acid, polymethacrylic acid, alginate, heparan sulfate, polyglutamic acid, polyaspartic acid, poly(4-vinylbenzoic acid), hyaluronic acid, polystyrenesulfonate, heparin, chondroitin sulfate, dextran sulfate, polymethacrylic acid, carboxymethyl cellulose, polystyrene sulfonate, DNA, RNA, or a combination thereof.

Individual cells can be killed or detached from a surface of a fluidic device with light. Light may also be used to weaken adherence or binding of cells to a fluidic device surface to facilitate their removal during a subsequent wash step. The light may be selectively applied to individual cells within the fluidic device, for example by using an imaging device to identify locations of cells intended for removal and a spatial light modulator (SLM) such as a digital micromirror device (DMD) to direct the light to the locations of selected cells. Accordingly, the light may be applied only to cells that are intended for removal from the fluidic device, and may be selectively withheld from cells intended for further analysis or retention.

For example, certain aspects of the present application provide a method for separating a plurality of cells that comprises inputting the plurality of cells into a fluidic device, identifying a first cell of the plurality of cells to retain in the fluidic device and a second cell of the plurality of cells to remove from the fluidic device, irradiating the second cell with light and not irradiating the first cell with light, and removing the second cell but not the first cell from the fluidic device. In similar aspects, the present application provides a method for separating a plurality of cells that comprises inputting the plurality of cells into a channel of a fluidic device, identifying a first cell of the plurality of cells to retain in the channel and a second cell of the plurality of cells to remove from the channel, irradiating the second cell with light and not irradiating the first cell with light, and removing the second cell but not the first cell from the channel. The light may detach or weaken surface binding of the second cell, such that the second cell may be selectively removed in a mild wash step.

A light-mediated removal method can also include enclosing non-removed cells in one or more chambers. In one such aspect, the present application provides a method for separating a plurality of cells that comprises inputting the plurality of cells into a fluidic device; synthesizing one or more chambers in the fluidic device to enclose a first cell of the plurality of cells and not enclose a second cell of the plurality of cells; irradiating the second cell with light; and removing the second cell from the fluidic device; wherein the first cell remains in the one or more chambers following the removing. In a further aspect, the present application provides a method for separating a plurality of cells that comprises inputting the plurality of cells into a channel of a fluidic device; synthesizing one or more chambers in the channel to enclose a first cell of the plurality of cells and not enclose a second cell of the plurality of cells; irradiating the second cell with light; and removing the second cell from the channel; wherein the first cell remains in the one or more chambers following the removing. In many such cases, the first cell is not irradiated with light. For example, the light may be selectively applied to a location in the fluidic device that contains the first cell. The light may be applied to all cells that are not enclosed within chambers or to only a subset of cells that are not enclosed in chambers. The light may be applied vertically through an about 5 to 10, about 5 to 25, about 5 to 50, about 5 to 100, about 5 to 250, about 5 to 500, about 5 to 1000, about 5 to 2500, about 5 to 5000, about 5 to 10000, about 10 to 25, about 10 to 50, about 10 to 100, about 10 to 250, about 10 to 500, about 10 to 1000, about 10 to 2500, about 10 to 5000, about 10 to 10000, about 25 to 50, about 25 to 100, about 25 to 250, about 25 to 500, about 25 to 1000, about 25 to 2500, about 25 to 5000, about 25 to 10000, about 50 to 100, about 50 to 250, about 50 to 500, about 50 to 1000, about 50 to 2500, about 50 to 5000, about 50 to 10000, about 100 to 250, about 100 to 500, about 100 to 1000, about 100 to 2500, about 100 to 5000, about 100 to 10000, about 250 to 500, about 250 to 1000, about 250 to 2500, about 250 to 5000, about 250 to 10000, about 500 to 1000, about 500 to 2500, about 500 to 5000, about 500 to 10000, about 1000 to 2500, about 1000 to 5000, about 1000 to 10000, about 2500 to 5000, about 2500 to 10000, or about 5000 to 10000 μm² area of the fluidic device. The light may comprise a sufficient dosage of ultraviolet (UV) light to kill the second cell, or to detach the second cell from a surface of the channel of the fluidic device. The light may also diminish an affinity of the second cell for a surface of the channel or an adherent support coupled to the surface of the channel.

An example of a light-based cell removal method is provided in FIG. 13, which depicts a fluidic device channel (1300) that comprises a collection of cells (1301) disposed on a cell adherent support (1303) attached to a first surface (1302) of the channel (1300). A subset of the cells (1301) can be enclosed (1310) in chambers (1311). Light (1321) can then be applied (1320) to one or more cells that are not enclosed within chambers (1322). The light (1321) can detach the one or more cells to which it is applied (1322) from the cell adherent support (1303), facilitating removal of these cells in a subsequent wash step (1330).

Cells can also be removed from a fluidic device by stripping a support to which they are adhered. For example, a method can include inputting a plurality of cells into a fluidic device comprising a first surface comprising a cell adherent support; wherein a first cell is disposed on (e.g., settled on or adhered to) a first portion of the cell adherent support and a second cell is disposed on a second portion of the cell adherent support; detaching the second portion of the cell adherent support from the first surface of the fluidic device, thereby detaching the second cell from the surface of the fluidic device; and removing the second cell from the fluidic device. Similarly, a method can include inputting a plurality of cells into a channel of a fluidic device comprising a first surface comprising a cell adherent support; wherein a first cell is disposed on (e.g., settled on or adhered to) a first portion of the cell adherent support and a second cell is disposed on a second portion of the cell adherent support; detaching the second portion of the cell adherent support from the first surface of the channel, thereby detaching the second cell from the surface of the channel; and removing the second cell from the channel. In many cases, the removing includes a wash step, wherein the first cell remains adhered to the first portion of the cell adherent support coupled to the surface of the flow cell. Accordingly, the method can include retaining the first cell following the removal of the second cell.

Such a method may comprise enclosing a cell within one or more chambers. For example, a method for separating a plurality of cells can include inputting the plurality of cells into a fluidic device comprising a first surface comprising a cell adherent support; synthesizing one or more chambers in the fluidic device to enclose a first cell of the plurality of cells and not enclose a second cell of the plurality of cells, wherein the second cell is bound to a first portion of the cell adherent support; detaching at least the first portion of the cell adherent support from the first surface of the fluidic device, thereby detaching the second cell from the surface of the fluidic device; and removing the second cell from the fluidic device. Similarly, a method for separating a plurality of cells can include inputting the plurality of cells into a channel of a fluidic device comprising a first surface comprising a cell adherent support; synthesizing one or more chambers in the channel to enclose a first cell of the plurality of cells and not enclose a second cell of the plurality of cells, wherein the second cell is bound to a first portion of the cell adherent support; detaching at least the first portion of the cell adherent support from the first surface of the channel, thereby detaching the second cell from the surface of the channel; and removing the second cell from the channel. The method can include retaining the first cell following the removal of the second cell. In particular, the first cell can remain enclosed in the one or more chambers following this removal step.

Furthermore, when the first cell is adhered to a second portion of the cell adherent support, for example a portion of the cell adherent support that is co-enclosed with the first cell in the one or more chambers, the second portion of the cell adherent support may remain coupled to the first surface of the channel following detachment of the first portion of the cell adherent support from the first surface of the channel. The detachment may be spatially targeted, for example through application of light or a chemical reagent the first portion and not to the second portion of the cell adherent support. Similarly, the detachment may be effectuated by a species that can contact the first portion of the cell adherent support but is unable to access the second portion of the cell adherent support. When the second portion of the cell adherent support is disposed within one or more chambers, the detachment may be mediated by a species that is unable to diffuse into the one or more chambers. For example, the detachment may include cell adherent support displacement by a multi-kilodalton or larger polyionic species (e.g., a polyanion such as polyacrylic acid) that is larger than pores of walls of the one or more chambers, and therefore is unable to permeate into the one or more chambers.

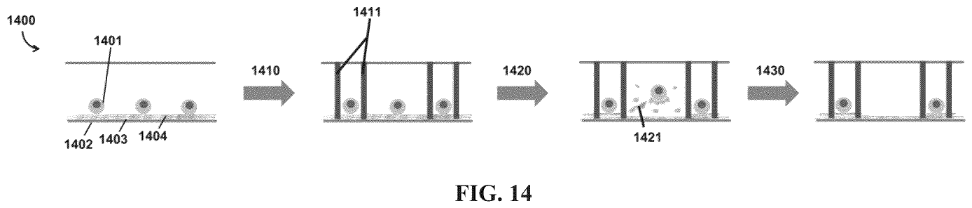

An example of this method is provided in FIG. 14, which illustrates a plurality of cells (1401) disposed on a cell adherent support (1404) in a channel of a fluidic device (1400). The cell adherent support (1404) is coupled to an additional support (1403) on the bottom surface of the fluidic device channel (1402). A first cell can be enclosed (1410) in one or more chambers (1411). An interstitial portion of the cell adherent support (1421) can be detached (1420) from the additional support (1403), causing an interstitial cell disposed on the interstitial portion of the cell adherent support (1421) to detach from the bottom surface of the fluidic device channel (1402), and enabling its removal in a subsequent wash step (1430) that does not remove chamber-enclosed cells from the fluidic device channel.

In some aspects, the cell adherent support is detached from the fluidic device surface with a polyionic species. Cell adherent supports that are noncovalently adhered to a fluidic device surface or to an additional support coupled to the surface of the fluidic device may be displaced by an ionic species, and in particular by ionic species with similar charge profiles. Many of the cell adherent supports disclosed herein comprise one or more polyanionic components of extracellular matrices (e.g., fibronectin, laminin, and the like), and can likewise be displaced by polyanions. For example, the cell adherent support may comprise a polyanionic extracellular matrix component coupled to a polycationic (e.g., poly-l-ornithine) coating on the first surface, and the polyanionic extracellular matrix component may be displaced by a polyanion input into the fluidic device. In many cases, the polyanion displaces at least a portion of the cell adherent support and then couples to the first surface. Examples of polyanions suitable for cell adherent support detachment include polyacrylic acid, polymethacrylic acid, alginate, heparan sulfate, polyglutamic acid, polyaspartic acid, poly(4-vinylbenzoic acid), hyaluronic acid, polystyrenesulfonate, heparin, chondroitin sulfate, dextran sulfate, polymethacrylic acid, carboxymethyl cellulose, polystyrene sulfonate, DNA, RNA, as well as copolymers and combinations of these polymers. In particular cases, the polyanion includes polyacrylic acid, polymethacrylic acid, alginate, heparan sulfate, polyglutamic acid, polyaspartic acid, poly(4-vinylbenzoic acid), hyaluronic acid, polystyrenesulfonate, heparin, chondroitin sulfate, dextran sulfate, polymethacrylic acid, carboxymethyl cellulose, or a copolymer of combination thereof. The polyanion can comprise a molecular weight of about 0.5 to 1 kilodaltons (kDa), about 0.5 to 2.5 kDa, about 0.5 to 5 kDa, about 0.5 to 10 kDa, about 0.5 to 25 kDa, about 0.5 to 50 kDa, about 0.5 to 100 kDa, about 0.5 to 250 kDa, about 0.5 to 500 kDa, about 0.5 to 1000 kDa, about 0.5 to 2500 kDa, about 0.5 to 5000 kDa, about 0.5 to 10000 kDa, about 1 to 2.5 kDa, about 1 to 5 kDa, about 1 to 10 kDa, about 1 to 25 kDa, about 1 to 50 kDa, about 1 to 100 kDa, about 1 to 250 kDa, about 1 to 500 kDa, about 1 to 1000 kDa, about 1 to 2500 kDa, about 1 to 5000 kDa, about 1 to 10000 kDa, about 2.5 to 5 kDa, about 2.5 to 10 kDa, about 2.5 to 25 kDa, about 2.5 to 50 kDa, about 2.5 to 100 kDa, about 2.5 to 250 kDa, about 2.5 to 500 kDa, about 2.5 to 1000 kDa, about 2.5 to 2500 kDa, about 2.5 to 5000 kDa, about 2.5 to 10000 kDa, about 5 to 10 kDa, about 5 to 25 kDa, about 5 to 50 kDa, about 5 to 100 kDa, about 5 to 250 kDa, about 5 to 500 kDa, about 5 to 1000 kDa, about 5 to 2500 kDa, about 5 to 5000 kDa, about 5 to 10000 kDa, about 10 to 25 kDa, about 10 to 50 kDa, about 10 to 100 kDa, about 10 to 250 kDa, about 10 to 500 kDa, about 10 to 1000 kDa, about 10 to 2500 kDa, about 10 to 5000 kDa, about 10 to 10000 kDa, about 25 to 50 kDa, about 25 to 100 kDa, about 25 to 250 kDa, about 25 to 500 kDa, about 25 to 1000 kDa, about 25 to 2500 kDa, about 25 to 5000 kDa, about 25 to 10000 kDa, about 50 to 100 kDa, about 50 to 250 kDa, about 50 to 500 kDa, about 50 to 1000 kDa, about 50 to 2500 kDa, about 50 to 5000 kDa, about 50 to 10000 kDa, about 100 to 250 kDa, about 100 to 500 kDa, about 100 to 1000 kDa, about 100 to 2500 kDa, about 100 to 5000 kDa, about 100 to 10000 kDa, about 250 to 500 kDa, about 250 to 1000 kDa, about 250 to 2500 kDa, about 250 to 5000 kDa, about 250 to 10000 kDa, about 500 to 1000 kDa, about 500 to 2500 kDa, about 500 to 5000 kDa, about 500 to 10000 kDa, about 1000 to 2500 kDa, about 1000 to 5000 kDa, about 1000 to 10000 kDa, about 2500 to 5000 kDa, about 2500 to 10000 kDa, or about 5000 to 10000 kDa. In some cases, the polyanion comprises a molecular weight of at least about 0.5 kDa, about 1 kDa, about 2.5 kDa, about 5 kDa, about 10 kDa, about 25 kDa, about 50 kDa, about 100 kDa, about 250 kDa, about 500 kDa, about 1,000 kDa, about 2500 kDa, or about 5000 kDa. In some cases, the polyanion comprises a molecular weight of at most about 1 kDa, about 2.5 kDa, about 5 kDa, about 10 kDa, about 25 kDa, about 50 kDa, about 100 kDa, about 250 kDa, about 500 kDa, about 1,000 kDa, about 2,500 kDa, about 5,000 kDa, or about 10,000 kDa. In many cases, the polyanion is prohibitively large to diffuse through walls of the one or more chambers, such that walls of the one or more chambers are impermeable to the polyanion. In such cases, the polyanion may comprise a molecular weight of about 50 to 1000 kDa, about 100 to 2500 kDa, or about 100 to 5000 kDa.

In some cases, a polycation may be used to detach a cell adherent support. Examples of polycations suitable for this purpose include chitosan, poly-l-ornithine, and poly-l-lysine. The polycation may be sized similarly to a polyanion disclosed herein, for example comprising a molecular weight of about 0.02 to 10000 kDa.

A cell adherent support can be detached from the first surface of the fluidic device using light. The cell adherent support may be coupled to the surface of the fluidic device or to an additional support coupled to the surface of the fluidic device through a light-cleavable linker. The cell adherent support may also include light-cleavable groups at internal positions, such that light may be used to degrade the cell adherent support in selected regions of the fluidic device.

A cell adherent support may also be detached from the first surface of the fluidic device with a hydrolytic enzyme such as a protease. While in many aspects disclosed herein, enzymes are added at concentrations that insubstantially degrade and detach cell adherent supports from fluidic device surfaces, in some aspects disclosed herein, an enzyme may be added at a concentration and for a duration suitable for cell adherent support detachment.

A disclosed method can include determining a characteristic of the first cells and optionally the second cells prior to, during, or following the removal of the second cells from the fluidic device channel. In many cases, the methods disclosed herein include determining a characteristic of the first cells following the removal of the second cells from the fluidic device channel. The assays disclosed herein can comprise a chemical, biochemical or molecular reaction (such as a cleavage of a bond, specific binding of complementary components, enzymatic reactions, dissolution of complementary components, or the like) or a change of physical state (such as an increase or decrease in temperature, change in energy level, or the like), along with a measurement of such reaction or change in physical state. The signals generated during an assay can include, but are not limited to, an electrical signal, an optical signal, a chemical signal, or a material output. A material output comprises the production of a material that contains information that can be decoded or extracted. For example, a material output may be the amplification of a polynucleotide whose length, quantity, composition, or nucleotide sequence is indicative of a cellular characteristic. Characteristics or properties of cells that are detected or measured can include cytotoxicity, viability, proliferation capacity under selected conditions, size, shape, motility, types and profiles of cell surface, or cell membrane proteins, types and profiles of secreted proteins, production of metabolites, transcriptome, gene copy numbers, gene or allele identity, chromatin accessibility profiles, vector copy numbers for engineered or infected cells, and the like. Additional assays may include culture contamination assays including, but not limited to, viral, bacterial, yeast, mold, or mycoplasma assays, endotoxin assays, and cellular morphology assays.

A method disclosed herein can include detecting a guide ribonucleic acid (RNA) associated with a genetic modification of the first cells and/or the second cells. The cells can be transiently or stably transfected with a sequence encoding a guide RNA specific for a particular genomic sequence. The guide RNA can be coupled to a barcode, an exogenous messenger RNA (e.g., a selection marker), a capture sequence (e.g., a polyA tail), or a combination thereof. The first cell or the aggregate of the first cells can express a Cas protein that can utilize the guide RNA. Alternatively, a Cas protein can be delivered to the cells (e.g., in a liposome). Cell growth or movement can then be correlated with a genomic edit imparted by a particular guide RNA sequence. In particular, the cells can be lysed to release guide RNA. The guide RNA can optionally be captured, and then be used as a template for generating a cDNA molecule comprising a complement of the guide RNA sequence, and optionally additional sequences coupled to the guide RNA such as the exogenous mRNA, the barcode, or a combination thereof. The cDNA molecule can be coupled to a spatial location tag corresponding to a unique location within the channel of the fluidic device. For example, the spatial location tag can be present in a capture probe that hybridizes to the guide RNA and serves as a reverse transcription primer, and the method of generating the cDNA molecule can include capturing the guide RNA on the capture probe comprising the spatial location tag or the complement thereof, and reverse transcribing the guide RNA on the capture probe, thereby generating the cDNA molecule. The method can include sequencing the cDNA, thereby detecting the guide RNA associated with the genetic modification of the cells.

A method can include determining an alternative or additional characteristic of the first cells and/or the second cells. As an example, the characteristic can be an mRNA expressed by the cells, which may optionally be determined by lysing the first cell or at least a subset of the aggregate of the first cells, capturing the mRNA on a capture element (e.g., a nucleic acid capture probe) coupled to a surface of the flow cell, reverse transcribing the mRNA to generate a cDNA molecule that optionally comprises a barcode sequence derived from the capture element, and sequencing the cDNA molecule. In many cases, mRNA analysis comprises capturing mRNAs from the cells enclosed within a chamber, pathway, or obstacle course. The one or more capture elements can be coupled to a surface of the fluidic device. The fluidic device containing the cells may be loaded with a lysing reagent so that messenger RNAs of the cells or cell aggregates are released and captured by the capture elements, and with reverse transcription reagents to copy the captured oligonucleotide labels to produce complementary DNAs thereof. The complementary DNAs may then be eluted from the fluidic device (e.g., cleaved from a surface of the top or bottom layer) and sequenced. It is understood that a sequencing step may comprise additional steps in particular embodiments including, but not limited to, tagmentation, adding adaptors, cleaving the cDNA to form appropriate lengths for sequencing, and the like. In some embodiments, an additional step may be implemented for depolymerizing or degrading the polymer matrix walls of the chambers after mRNA capture. Reverse transcription reagents comprise conventional reagents for reverse transcription; namely, a reverse transcriptase (such as, a Moloney murine leukemia virus (MMLV)), dNTPs, optional RNAse inhibitor, buffer. The sequencing step may be carried out at the sites of the captured mRNAs (in situ) or cDNAs may include a spatial barcode and be eluted and sequenced on a separate sequencing instrument (“external” sequencing). For in situ sequencing, further steps may include (i) amplifying the complementary DNAs, e.g. by bridge amplification, or like method, (ii) sequencing the amplified complementary DNAs, e.g. by a sequencing-by-synthesis technique, and (iii) determining relative expression of the mRNAs for the cells of each of the chambers. For external sequencing further steps may include (i) providing capture elements comprising spatial barcodes, (ii) synthesizing cDNAs comprising spatial barcodes, and (iii) eluting and sequencing the cDNAs and correlating each cDNA with a chamber location by its spatial barcode.

The characteristic can also be a soluble factor secreted by the first cells and/or the second cells. The soluble factor may be detected by disposing a capture surface comprising an affinity reagent that binds the soluble factor adjacent to the cells, and detecting the soluble factor bound to the capture surface. Such a method can include disposing a capture surface comprising an affinity reagent that binds the soluble factor adjacent to the cells and detecting the soluble factor bound to the capture surface. In some cases, the capture surface is enclosed with the first cells within one or more chambers. In such cases, the capture surface can be loaded into the fluidic device channel prior to chamber synthesis to be co-enclosed with the first cells in the one or more chambers. In other cases, the capture surface is disposed outside of the one or more chambers. The capture surfaces can be in proximity to the one or more chambers enclosing the one or more cells and/or in proximity to the second cells outside of the one or more chambers. The capture surface can be loaded into the fluidic device at a controlled density, for example 1 capture surface per about 100, 50, 10, 5, 1, 0.5, 0.1, 0.05, 0.01, 0.005, or 0.001 mm² of the fluidic device.

In an exemplary embodiment, the capture surface comprises a bead. As used herein, the term “bead” can denote a microparticle or a nanoparticle, such as a ceramic, metal, metal oxide, polymer, or saccharide-based 30 to 10000 nm particle. However, further capture surfaces, including nanotubes, nucleic acid nanostructures, and antibody Fc domains. The capture surface affinity reagent can, as non-limiting examples, include antibodies, antibody fragments, aptamers, affimers, or a combination thereof.

In a further exemplary embodiment, the soluble factor comprises a cytokine such as interferon-γ (IFN-γ) and interferon-α (IFN-α), an interleukins such as interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-13 (IL-13), interleukin-15 (IL-15), interleukin-21 (IL-21), or interleukin-23 (IL-23), a colony stimulating factor (CSFs) such as granulocyte-macrophage colony stimulating factor (GM-CSF), granulocyte colony stimulating factor (G-CSF), or a tumor necrosis factors (TNF) such as TNF-α or TNF-β. In another embodiment, the secreted factor comprises an effector molecule such as a granzyme.

A soluble factor bound to a capture surface (e.g., an affinity reagent of a capture surface) can be detected by contacting the soluble factor bound to said capture surface with a labeled antibody configured to bind to the soluble factor, and detecting the labeled antibody. Multiple soluble factors can be detected in a single assay by providing a capture surface or plurality of capture surfaces that comprise a plurality of affinity reagents configured to bind the plurality of soluble factors, contacting the plurality of soluble factors bound to the capture surface or plurality of capture surfaces with a plurality of labeled antibodies configured to bind to the plurality of soluble factors, and detecting a plurality of labels coupled to the plurality of antibodies. In this way, 2, 3, 4, 5, 6, or more soluble factors can be detected in a single assay. For example, an assay may utilize 2, 3, 4, 5, 6, or more beads that each include a different affinity reagent configured to bind to a different soluble factor and a commensurate number of antibodies configured to bind to the soluble factors and optionally containing distinguishable detectable labels (e.g., different fluorophores or oligonucleotide barcodes). In many cases, the soluble factor is a protein or a metabolite, such as a cytokine, immune active protein, hormone, or neurotransmitter.

In some cases, the first cells comprise an effector cell, and the characteristic comprises cytotoxicity. In such cases, determining the cytotoxicity of the first cells comprises measuring a rate or an occurrence of the effector cell killing target cells. The target cells can be enclosed with the first cells in the one or more chambers. The target cells may be a subset of the second cells that are enclosed in the one or more chambers. To prevent interstitial target cells from affecting cytotoxicity measurements, second cells that are not enclosed by the one or more chambers can be removed from the channel using one or more of the strategies disclosed herein. The second cells may comprise a sample of tumor cells from a patient or a cell line such as hepatic tumor cell line SK-HEP-1, Chava et. al., J. Vis. Exp., 2020 Feb. 22: (156): 10.3791/60714. Examples of effector cells include Tc1 cells, Tc2 cells, Tc9 cells, Tc17 cells, Tc22 cells, and natural killer cells. In some embodiments, the effector cell is engineered for a therapeutic purpose, such as expression of a chimeric antigen receptor that confers cytotoxicity against a particular cancer cell.

The characteristic can also include activation. Cellular activation can be measured with numerous techniques disclosed herein, including surface marker expression, soluble factor secretion, transcriptomic analysis, proliferation, morphology, or a combination thereof. The disclosed methods are broadly amenable to detecting activation caused by contact between two or more cells or intercellular signaling mediated by secreted soluble factors. In a particular aspect of the present disclosure, determining activation comprises detecting a cell surface marker. For example, the method can comprise contacting the first cell or aggregate of the first cells with a binding agent configured to bind to the surface marker and detecting the binding agent. Activation can also be determined by detecting expression of a surface marker, a morphology change, a cytotoxicity increase, a proliferation rate change, a soluble factor, a genomic sequence, an mRNA, or a combination thereof of the first and/or second cells.

In some aspects, determining a characteristic of the first and/or second cells includes determining a proliferation rate of the cells. As used herein, the term “proliferation” refers to cell division and development processes that increase cell number. It is understood that the term “proliferation rate” may include a measure of a lack of proliferation. For example, chambers enclosing one or more cells may be exposed to an agent (e.g., a drug candidate) to determine whether the agent promotes, kills, or retards the growth of the cells, for example in comparison to controls not exposed to the agent. Thus, in the case of the treated cells, a negative “proliferation rate” may be possible because the final numbers of cells counted in the chambers may be less than the original numbers; or a signal monotonically related to cell number may decline in value. In some embodiments, cells may be stained with a membrane or intracellular dye for determining proliferation by dye dilution so that an independent measure of cell proliferation may be obtained. Exemplary intracellular dyes for dye dilution include, but are not limited to, Hoechst 33342, carboxyfluorescein succinimidyl ester (CFSE), and the like. If a subpopulation of interest is present as only a small fraction of a total population then a larger number chambers is required.

In some aspects, determining a characteristic of the first and/or second cells includes detecting a surface marker of the cells. As used herein, the term “surface marker” denotes species that are expressed on the surface of a cell. Exemplary surface markers include surface proteins such as G protein-coupled receptors (GPCRs), ion channels, engineered receptors (e.g., chimeric antigen receptors), and cluster of differentiation (CD) molecules (e.g., CD3, CD4, CD5, CD6, CD7, CD8, etc.), as well as carbohydrates (e.g., sialic acids), glycolipids, and the like. However, in particular aspects of the present disclosure, the surface markers are proteins.

Surface marker detection can include contacting cells with a binding agent configured to bind to the surface marker and detecting the binding agent, thereby detecting the surface marker. A single surface marker or plurality of surface markers can be detected in a single assay. More precisely, the binding agent can include a single binding agent configured to bind to a single surface marker or a plurality of binding agents configured to bind to a plurality of surface markers. Multiple surface markers can be detected simultaneously, for example by contacting the cells with multiple antibodies that bind different surface markers and are coupled to distinct detectable labels such as fluorophores that are simultaneously detectable on separate imaging channels. Alternatively or in addition thereto, two or more surface markers can be detected sequentially, for example by contacting the cells with a first antibody that binds to a first surface marker, detecting the first antibody, binding the cells with a second antibody that binds to a second surface marker, and detecting the second antibody. In particular cases, detecting a plurality of surface markers includes detecting relative expression levels of the plurality of surface markers. Exemplary binding agents include antibodies; antibody fragments such as single-chain antibody molecules, scFvs, Fab domains, diabodies, nanobodies, minibodies, linear antibodies, and cross-Fab fragments; aptamers; and affimers. A binding agent may be coupled to a detectable label such as a fluorescent label, an oligonucleotide label, a colorimetric label, an enzymatic label (e.g., pyrophosphatase), or a combination thereof. It is noted that a binding agent such as an antibody can be coupled to a cell prior to or following the cell's introduction into a channel and/or encapsulation within a chamber.

In some aspects, determining a characteristic of the first and/or second cells includes identifying a morphological feature of the cells. The characteristic may be identified, for example, by imaging the cells. In some cases, the feature is identified with brightfield imaging. However, morphological features can also be identified with fluorescence imaging, which may utilize dyes that localize, partition, or intercalate into specific cellular structures such peroxisomes or lipid membranes. In some cases, the morphological feature is flattening or spread associated with adherence to a surface.

The disclosed methods are applicable to a broad number of cell types. As non-limiting examples the first cells, the second cells, or a combination thereof can comprise an adipocyte, a cardiomyocyte; chondrocyte, an ectoderm, an embryonic stem cell, an endodermal cell, an endothelial cell, a fibroblast, a hematopoietic cell, a hematopoietic stem cell, a satellite, a hepatocyte, an islet cell, a keratinocyte, a mesenchymal cell, a mesenchymal stem cell, a progenitor cell, a myoblast, a myocyte, a neural cell, an oligodendrocyte, an osteoblast, a pancreatic epithelial cell, a skeletal myocyte cell, a smooth muscle cell, or a white blood cell.

In some cases, the first cells comprise an adherent cell. In other cases, the first cells comprise a non-adherent cell. In some cases, the second cells comprise an adherent cell. In other cases, the second cells comprise a non-adherent cell. In some cases, the first cells and the second cells comprise the same type of cell. In other cases, the first cells and the second cells are different cell types.

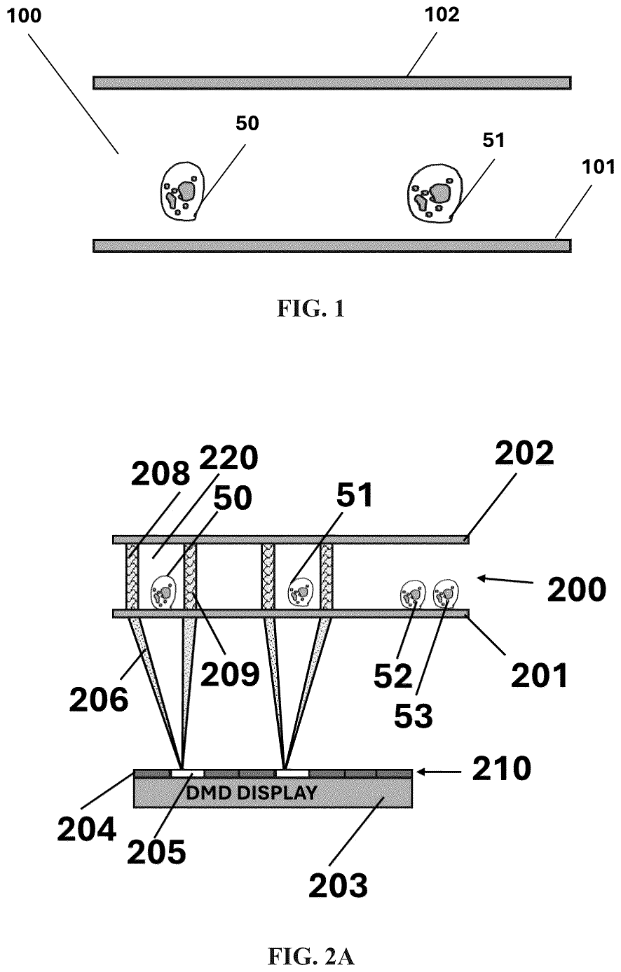

A channel or chamber of a fluidic device (also sometimes referred to as a “flow chamber,” “flow channel,” or “reaction chamber”) may receive or be configured to receive a biological sample. FIG. 1 shows a simplified schematic cross-sectional side view illustration of a portion of the channel 100 of the fluidic device disclosed herein. The channel 100 may comprise a first surface 101 and a second surface 102. In some embodiments, the first surface 101 and the second surface 102 are disposed, placed, or positioned opposite of one another (e.g., as depicted in FIG. 1). For example, the first surface can be provided by the bottom layer and the second surface can be provided by the top layer that at least partially define the channel. In some embodiments, a middle spacer layer of double-sided adhesive with a cut-out portion can be used to position the first surface 101 and second surface 102 in a facing relationship to at least partly form the flow channel. As a non-limiting example, the cut-out portion can have a dimension of about 0.7 cm width and about a 10 cm length. In some embodiments, the first surface and second surface are substantially parallel, so that the perpendicular distance between them is substantially the same throughout the channel, for example, where chambers are formed. In some embodiments, the perpendicular distance between a first surface and a second surface depends in part on the nature and size of the biological components to be analyzed. In some embodiments, such as, those adapted to analyzing mammalian cells, the perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the biological component to be analyzed to five times the average size of the biological component to be analyzed. In some embodiments, the perpendicular distance between a first surface and a second surface may be in the range of from twice the average size of the largest biological component in the biological sample to five times the average size of the largest biological component in the biological sample. In some embodiments, the first surface 101 may be a lower surface. In certain embodiments, the second surface 102 may be an upper surface. The channel 100 may receive a biological sample comprising one or more biological components 50, 51. The channel 100 may receive one or more polymer precursors. As illustrated in FIG. 1, the biological components 50, 51 may include cells. However, as discussed herein, the biological components may include tissues, proteins, nucleic acids, etc. In some embodiments, the first surface 101, the second surface 102, or both surfaces may couple or receive, or be configured to couple or receive, at least one of the one or more biological components 50, 51. In some cases, the first surface 101 may couple or receive, or be configured to couple or receive, a biological component (e.g., biological components 50, 51). In certain cases, the second surface, 102 may couple or receive, or be configured to couple or receive, a biological component (e.g., biological components 50, 51). In some embodiments, the first surface and/or second surface can be optically transmissive so that visible and UV light can transmit through one or both of the surface for the generation of polymeric hydrogels, imaging of the flow cell, and the measurement of the analyte and biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

The first surface 101, the second surface 102, or both surfaces 101, 102 may be functionalized, for example with a coating. As a non-limiting example, a surface coating may be a surface polymer. Some non-limiting examples of surface coatings may include a capture reagent (e.g., pyridinecarboxaldehyde (PCA)), a functional group to capture one or more moieties (e.g., a chemical moiety), an acrylamide, an agarose, a biotin, a streptavidin, a strep-tag II, a linker, a functional group comprising an aldehyde, a phosphate, a silicate, an ester, an acid, an amide, an alkyne, an azide, an aldehyde dithiolane, or a combination thereof. In various embodiments, the surface coating may include a functional group to capture one or more moieties. For example, the acrylamide, the agarose, etc. may include such a functional group. In certain embodiments, the surface polymer may comprise polyethylene glycol (PEG), a thiol, an alkene, an alkyne, an azide, or combinations thereof. In various embodiments, the surface polymer may comprise a silane polymer. In some embodiments, the surface polymer may be functionalized with at least one of an oligonucleotide, an antibody, a cytokine, a chemokine, a protein, an antibody derivative, an antibody fragment, a carbohydrate, a toxin, or an aptamer. In particular embodiments, the surface coating comprises a material for which adherent cells have a binding affinity, such as fibronectin or laminin.

In some cases, the first surface 101, the second surface 102, or both surfaces 101, 102 may comprise one or more barcodes (e.g., nucleic acid barcodes or nucleic acid capture probes). In some embodiments, the first surface 101, the second surface 102, or both surfaces 101, 102 may comprise 1, 2, 3, 4, 5, 6, 7, 8, 9, 10, 15, 20, 30, 40, 50, 100, 200, 300, 400, 500, 600, 700, 1,000, 10,000, 50,000, 100,000, 250,000, 500,000, 1,000,000, 2,000,000, 5,000,000, 10,000,000, 15,000,000 barcodes, or any number of barcodes between any of the two numbers mentioned herein. The barcodes may collectively cover an area of about 500 nm² to about 5000 mm² of the first or second surface. In some embodiments, the first surface 101, the second surface 102, or both surfaces 101, 102 may comprise at most about 10,000,000 total number of barcodes. The barcodes may be different from one another (e.g., each barcode may be unique). In certain embodiments, a first portion or subset of the barcodes may be different from a second portion or subset of the barcodes. There may be 2, 3, 4, 5, 10, 15, 20, 25, 50, 75, 100, 1,000, 10,000 portions or subsets of the barcodes, or any number of portions or subsets of the barcodes between any of the two numbers mentioned herein. In some cases, a barcode (or a portion/subset of barcodes) may be associated with the location of the barcode on a surface (location coordinates (e.g., x-, y-coordinates) on a surface of a channel). In particular, a barcode may comprise a sequence that is unique (“spatially-addressed”) to a spot or region along a surface of the fluidic device. Each spot may comprise a plurality of nucleic acid barcodes that share this “spatially-addressed” sequence. Each barcode within a spot may additionally comprise a unique molecular identifier to normalize sequence counts within populations of amplicons generated using the fluidic device barcodes. Accordingly, the spot or region of origin of a barcode may be determined based on its spatially-addressed sequence. A spot or region that contains barcodes with common “spatially-addressed” sequences may comprise an area of about 10 to 10⁵ μm² along the first or second surface of the fluidic device. The first or second surface of the fluidic device may comprise about 50 to 500, about 50 to 1000, about 50 to 5000, about 50 to 10⁴, about 50 to 5×10⁴, about 50 to 10⁵, about 50 to 5×10⁵, about 50 to 10⁶, about 50 to 5×10⁶, about 50 to 10⁷, about 100 to 500, about 100 to 1000, about 100 to 5000, about 100 to 10⁴, about 100 to 5×10⁴, about 100 to 10⁵, about 100 to 5×10⁵, about 100 to 10⁶, about 100 to 5×10⁶, about 100 to 10⁷, about 500 to 1000, about 500 to 5000, about 500 to 10⁴, about 500 to 5×10⁴, about 500 to 10⁵, about 500 to 5×10⁵, about 500 to 10⁶, about 500 to 5×10⁶, about 500 to 10⁷, about 1000 to 5000, about 1000 to 10⁴, about 1000 to 5×10⁴, about 1000 to 10⁵, about 1000 to 5×10⁵, about 1000 to 10⁶, about 1000 to 5×10⁶, about 1000 to 10⁷, about 5000 to 10⁴, about 5000 to 5×10⁴, about 5000 to 10⁵, about 5000 to 5×10⁵, about 5000 to 10⁶, about 5000 to 5×10⁶, about 5000 to 10⁷, about 10⁴ to 5×10⁴, about 10⁴ to 10⁵, about 10⁴ to 5×10⁵, about 10⁴ to 10⁶, about 10⁴ to 5×10⁶, about 10⁴ to 10⁷, about 5×10⁴ to 10⁵, about 5×10⁴ to 5×10⁵, about 5×10⁴ to 10⁶, about 5×10⁴ to 5×10⁶, about 5×10⁴ to 10⁷, about 10⁵ to 5×10⁵, about 10⁵ to 10⁶, about 10⁵ to 5×10⁶, about 10⁵ to 10⁷, about 5×10⁵ to 10⁶, about 5×10⁵ to 5×10⁶, about 5×10⁵ to 10⁷, about 10⁶ to 5×10⁶, about 10⁶ to 10⁷, or about 5×10⁶ to 10⁷ spots or regions that contain barcodes with unique “spatially-addressed” sequences along one or more surfaces. Similarly, the fluidic device may comprise one or more fluidic channels, each of which may comprise about 50 to 10⁷ spots or regions that contain barcodes with unique “spatially-addressed” sequences along one or more surfaces.

A barcode may be attached to or coupled to the captured biological component. In some embodiments, the barcode may be a unique identifier that distinguishes a biological component from other biological components (e.g., that identifies a first biological component versus a second biological component). In some embodiments, a barcode may comprise a nucleic acid sequence (e.g., common sequence) to capture a biological component, or used in amplification. In some embodiments, a barcode may comprise a unique identifier comprising a unique nucleic acid sequence (e.g., DNA sequence, RNA sequence, etc.), protein tag, antibody, or an aptamer. In some embodiments the barcode may comprise a fluorescent molecule. In some embodiments, a location of the captured biological component may be associated with the unique identifier to, for example, retain spatial information of a biological component.

In some embodiments, the fluidic device may be a flow cell. For example, the fluidic device may be used for sequencing (e.g., DNA or RNA sequencing). In some embodiments, the fluidic device may be a microfluidic device. In certain embodiments, the fluidic device may be a nanofluidic device.

In particular embodiments, the present disclosure provides a fluidic device comprising a first surface comprising a first region comprising a cell adherent support and a second region comprising a non-adherent support. In some cases, the cell adherent support comprises actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof. In some cases, the second region is adjacent to the first region. In some cases, the non-adherent support comprises an acrylate, an acrylamide, an amine, a silane, a PEG, a PEG-silane, an acrylate-PEG-silane, a silane-PEG-silane, a PEG-amine, an acrylamide-PEG-amine, acrylate-PEG-amine, a COOH-PEG-amine, or a combination thereof. In some cases, the cell adherent support and non-adherent support are patterned over at least a portion of the first surface. In some cases, the first surface comprises a plurality of instances of the first region and a plurality of instances of the second region. In particular cases, the plurality of instances of the first region and the plurality of instances of the second region are tessellated over at least a portion of the first surface.

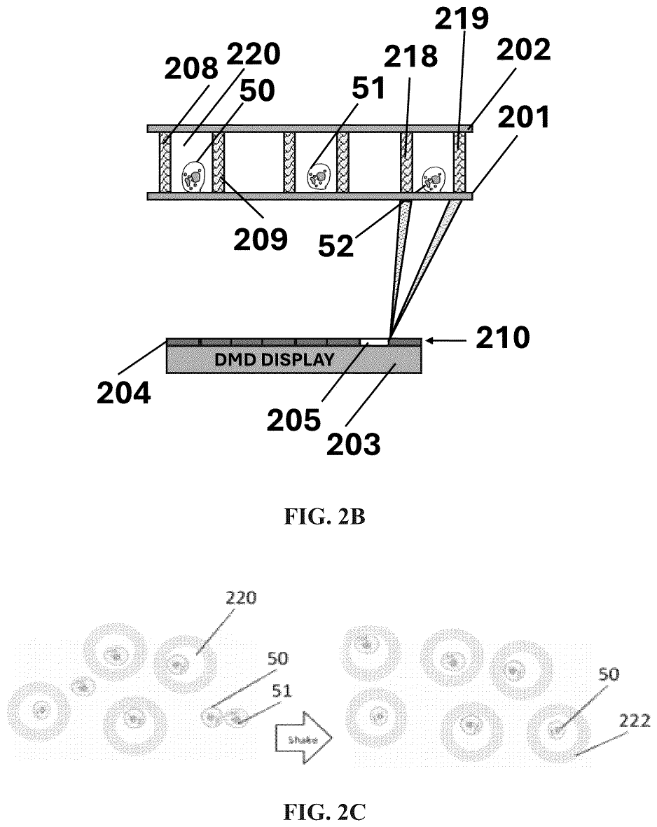

FIGS. 2A-C illustrate an exemplary method for forming a chamber enclosing one or more biological components in a fluidic device disclosed herein. FIG. 2A shows a portion of a system as provided herein (e.g., comprising a fluidic device as disclosed herein) including an energy source. FIG. 2B shows a polymer matrix being formed around a biological component in a portion of a system as provided herein. FIG. 2C shows a method of forming a polymer matrix around a biological component in a system as provided herein. As illustrated in FIGS. 2A-2C, in some embodiments, the one or more chambers extend from the bottom layer to the top layer of a fluidic device.

With continued reference to FIG. 2A, the channel 200 of the system may include a first surface 201 provided by the bottom layer and a second surface 202 provided by the top layer of the fluidic device. The energy source 203 may comprise one or more energy emitting portions (e.g., an energy emitting portion 205). In some embodiments, the energy source 203 may comprise one or more non-emitting portions (e.g., a non-emitting portion 204). The non-emitting portion 204 may not emit, or be configured to emit, energy. In some embodiments, the emitting portion 205 can emit energy in the form of electromagnetic waves (e.g., microwaves, light, heat, etc.) to at least a portion of the fluidic device. For example, the energy source may comprise an LED array in which individual LEDs can be selectively activated (e.g., act as an energy emitting portion 205) to create light projections with specified patterns. In some embodiments, the fluidic channel may be coupled to or disposed on a movable stage. In other embodiments, light may be projected to or onto at least a portion of the first fluidic channel to generate one or more polymer matrices. The light may be directed to various parts of the first fluidic channel. The energy source (e.g., light source) may be coupled to the fluidic device via an objective (e.g., a microscope objective or lens). The energy source may be directed to a portion of the fluidic channel (e.g., via a movable objective). In some cases, the light source, the objective, and/or the fluidic channel are movable to allow emission of energy to the fluidic channel so as to generate a pattern on at least a portion of a surface of the fluidic device. The polymer matrix may be formed similarly or complementary to the pattern of energy emission.

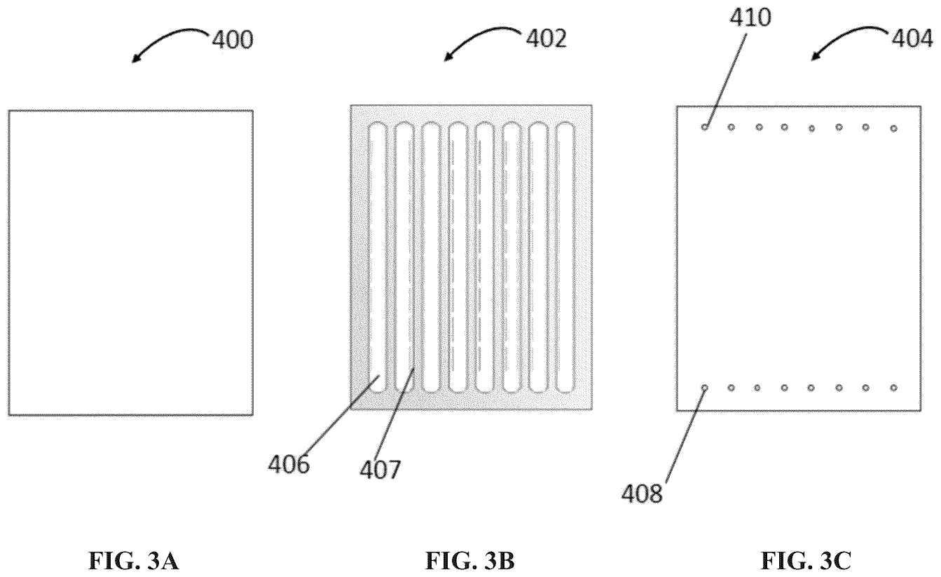

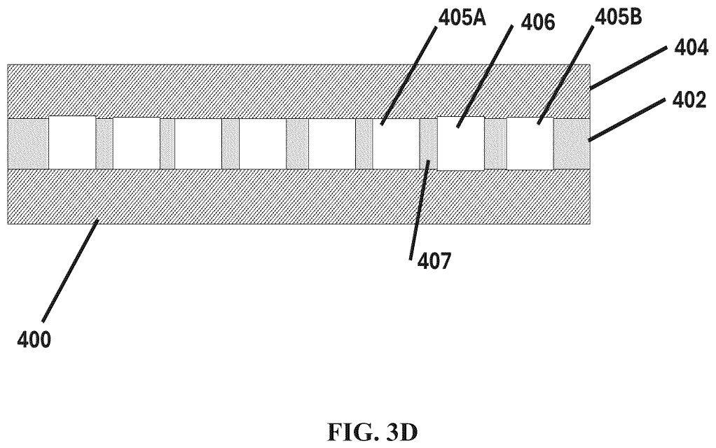

FIGS. 3A-D illustrate an example of a fluidic device consistent with the present disclosure. The channel 100 of FIG. 1 may correspond to any one of the cut-out regions (405A, 405B, 405C) of FIG. 3D. FIGS. 3A-D figures depict the components of a flow cell comprised of a bottom layer 400 (FIG. 3A), a spacer layer 402 (FIG. 3B), and top layer 404 (FIG. 3C). The bottom layer (400) and the top layer (404) can both independently be a glass or plastic material. In some cases, the top layer (404) is optically transparent or translucent. In some cases, the bottom layer (400) is optically transparent or translucent. In many cases, the top layer (404) and the bottom layer (400) are optically transparent or translucent. The spacer layer (402) can be a double-sided pressure sensitive adhesive with one or more cut-out regions (405A, 405B, 405C). In this design, the one or more cut-out regions of the spacer layer are sandwiched between the bottom layer and the top layer to form one or more channels. In various embodiments, the spacer layer (402) includes a core plastic (e.g., PET) layer with pressure sensitive adhesive coating on its top and/or bottom sides that contact the top (404) and bottom (400) layers. Examples of adhesive coatings consistent with the present disclosure include siloxane, silicone, acrylate, acrylamide, polyvinyl, polyurethane, epoxy, polyphenol, polyester, polyamide, polyimide, polytetrafluoroethylene, polyethylene, polypropylene, polycarbamate, polycarbonate, polyacrylic acid, sulfonated polyester, and combinations thereof.

Referring to FIG. 3B, a peripheral portion (407) of the spacer layer (402) provides a boundary for one or more cut-out regions (406) with defined widths and lengths. In many designs disclosed herein, the spacer layer (402) adheres to the top (404) and bottom (400) layers, such that a cut-out region or plurality of cut-out regions define a channel or a plurality of channels. The spacer layer (402), bottom layer (400), and/or top layer (404) can be water (or optionally more generally liquid) impermeable such that a first aqueous sample in a first cut-out region is isolated from a second aqueous sample in a second cut-out region. Biological components and reagents in a first cut-out region may similarly be isolated from other cut-regions. The one or more cut-out regions can thus define flow cell channels with defined dimensions.

A channel defined by a cut-out region can be operably coupled to an inlet (408) and/or an outlet (410) in the top layer (404), through which, for example, a gas, liquid, sample, or reagent may flow. In various embodiments, the inlet (408) and outlet (410) can both be represented as through holes in the top layer (404). However, the inlet (408) and/or outlet (410) can be disposed within other components of the flow cell, such as the bottom layer (400) or spacer layer (402).

With respect to FIG. 3D, which provides an end-on view of the fluidic device, the top layer (404) can have a height of about 0.7 mm and the bottom layer can have a height of about 0.5 mm. The cut-out region (406) can have a height defined by the distance between opposing faces of the top layer (404) and the bottom layer (400). The fluidic device can include multiple cut-out regions. For example, cut-out regions 405A and 405B may be disposed on opposite sides of cut out region 406. The cut-out regions may be identical or may have different shapes and/or dimensions. The spacer layer can have a height ranging from about 50 microns to about 200 microns, and preferably, from about 70 microns to about 130 microns. The cut-out region can have dimensions of about 10.3 cm length×about 0.7 cm width.

A channel of a fluidic device can comprise one or more polymer precursors for forming chambers. In some embodiments, the one or more polymer precursors are added to the fluidic device along with the one or more cells. In various embodiments, the one or more cells and the one or more polymer precursors can be pre-mixed and then added to the fluidic device at the same time; the one or more cells and the one or more polymer precursors can be added to the fluidic device at the same time; the one or more cells can be added to the fluidic device first and then the one or more polymer precursors can be added to the fluidic device second; or the one or more polymer precursors can be added to the fluidic device first and then the one or more cells can be added to the fluidic device second. Such precursors may be selected from a wide variety of compounds including, but not limited to, a diacrylate, a dimethacrylate, a diacrylamide, a dimethacrylamide, a diolefin, a polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or a combination thereof. In some embodiments, the hydrogel comprises an enzymatically degradable hydrogel, PEGthiol/PEG-acrylate, acrylamide/N,N′-bis(acryloyl)cystamine (BACy), or PEG/PPO. In some embodiments, the following precursors and crosslinker may be used to form chambers with degradable polymer matrix (hydrogel) walls. Polymer precursors may be formed by using any precursor and crosslinker of Table 3A (columns 1 and 3, respectively). The resulting polymer matrices may be degraded with the degradation agents listed in Table 3A (column 4). Representative crosslinkers useful for polymer synthesis are listed in Table 3B.

TABLE 3A

| Precursors | Hydrogels | Crosslinkers | Degradation Agents |
|---|---|---|---|
| Acrylamide | Polyacrylamide | Bis-acryloyl cystamine | DTT/TCEP/THP |
| | | (Structure 1) | |
| PEG-based acryloy1 | PEG | Bis(2- | DTT/TCEP/THP |
| | | methacryloly)oxyethyl | |
| | | disulfide (Structure 2) | |
| Dextran-based | Dextran | N,N′-(1,2- | NaIO₄ |
| acryloyl | | Dihydroxylethylene)bis- | |
| | | acrylamide (Structure 3) | |
| Polysacchride-base | Polysaccharide | Structure 4 | NaOH, |
| acryloyl | | | ethanolamine, |
| | | | DTT/TCEP/THP |
| Gelatin-base | Gelatin | Structure 5 | NaOH, |
| acryloyl | | | ethanolamine, |
| | | | nucleophilic bases |
| — | — | Structure 6 | NaOH, alkali, |
| | | | organic bases |
| — | — | Structure 7 | Acid |

TABLE 3B

| Structure | |
|---|---|
| Number | Formula |
| 1 | 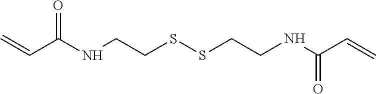 [SMILES: C=CC(=O)NCCSSCCNC(=O)C=C] |
| 2 | 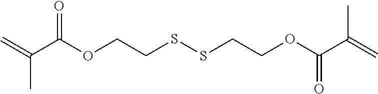 [SMILES: C=C(C)C(=O)OCCSSCCOC(=O)C(=C)C] |
| 3 | 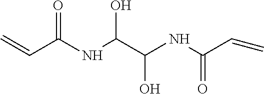 |
| 4 | 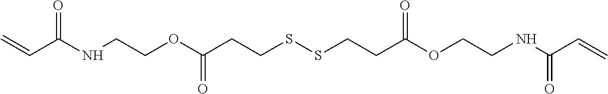 [SMILES: C=CC(=O)NCCOC(=O)CCSSCCC(=O)OCCNC(=O)C=C] |
| 5 | 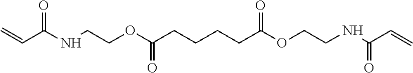 [SMILES: C=CC(=O)NCCOC(=O)CCCCC(=O)OCCNC(=O)C=C] |
| 6 | 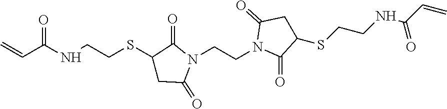 [SMILES: C=CC(=O)NCCSC1CC(=O)N(CCN2C(=O)CC(SCCNC(=O)C=C)C2=O)C1=O] |
| 7 | 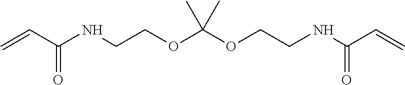 [SMILES: C=CC(=O)NCCOC(C)(C)OCCNC(=O)C=C] |
| 8 | 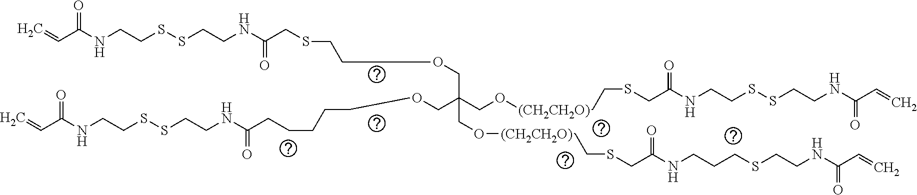 |
| | In some cases, n is an integer selected from 0-500. |
| | In some cases, n is an integer selected from 0-100. |
| | In some cases, n is an integer selected from 5-50. |
| 9 | 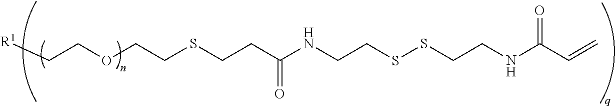 |
| | wherein R¹ is: |
| | i) a C₁ alkyl group, |
| | ii) a C₂-C₁₈ linear or branched saturated alkyl group, or |
| | iii) a C₃-C₈ cyclic saturated alkyl group substituted with 0-4 |
| | independently selected C₁-C₃ alkyl groups; |
| | wherein R¹ is substituted with q instances of |
| | 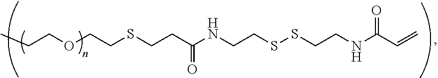 |
| | the remaining substituents on R¹ are hydrogen, q is an integer from 2 to 32, and n is an integer |
| | selected from 0-500. |
| | In some cases, q is an integer from 2 to 10. |
| | In some cases, q is an integer from 2 to 6. |
| | In some cases, q is an integer from 3 to 5. |
| | In some cases, R¹ is a C₁ alkyl group or a C₂-C₆ saturated linear or branched alkyl |
| | group, and q is an integer from 2-14. |
| | In some cases, R¹ is a C₁-C₂ alkyl group and q is an integer from 2 to 6. |
| | In some cases, n is an integer selected from 0-500. |
| | In some cases, n is an integer selected from 0-100. |
| | In some cases, n is an integer selected from 5-50. |
| 10 | 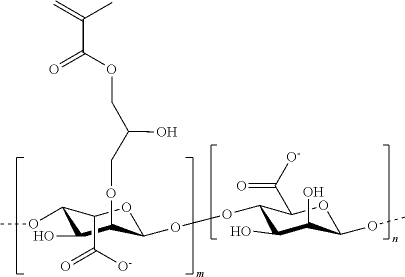 |
| | In some cases, n is an integer selected from 0-500. |
| | In some cases, n is an integer selected from 0-100. |
| | In some cases, n is an integer selected from 5-50. |
| | In some cases, m is an integer selected from 0-500. |
| | In some cases, m is an integer selected from 0-100. |
| | In some cases, m is an integer selected from 5-50. |
| 11 | 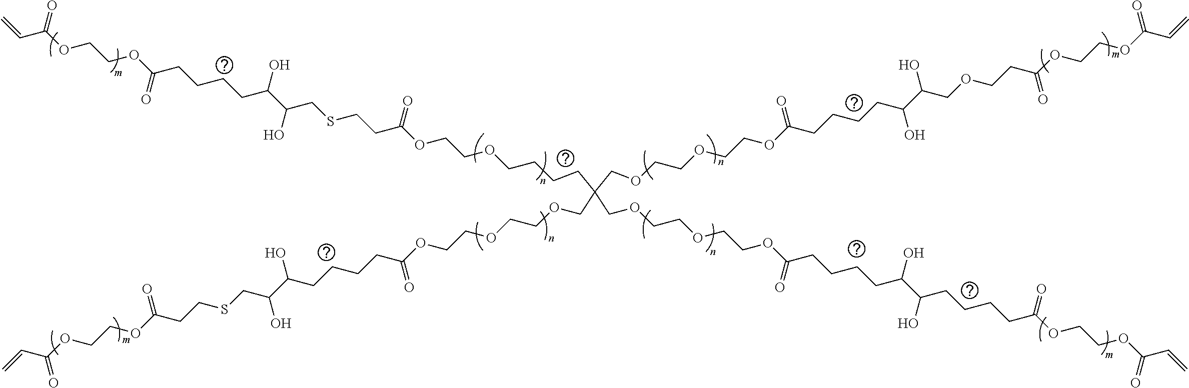 |
| | In some cases, n is an integer selected from 0-500. |
| | In some cases, n is an integer selected from 0-100. |
| | In some cases, n is an integer selected from 5-50. |
| | In some cases, m is an integer selected from 0-500. |
| | In some cases, m is an integer selected from 0-100. |
| | In some cases, m is an integer selected from 5-50. |
| 12 | 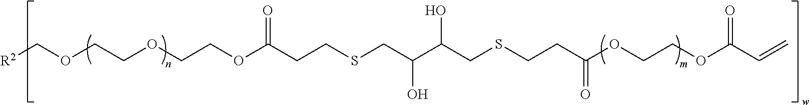 |
| | wherein R² is: |
| | i) a C₁ alkyl group, |
| | ii) a C₂-C₁₈ linear or branched saturated alkyl group, or |
| | iii) a C₃-C₈ cyclic saturated alkyl group substituted with 0-4 |
| | independently selected C₁-C₃ alkyl groups; |
| | wherein R² is substituted with w instances of |
| | 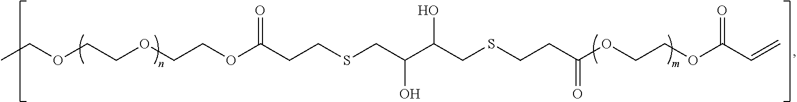 |
| | the remaining substituents on R² are hydrogen, w is an integer from 2 to 32, n is an |
| | integer selected from 0-500, and m is an integer selected from 0-500. |
| | In some cases, w is an integer from 2 to 10. |
| | In some cases, w is an integer from 2 to 6. |
| | In some cases, w is an integer from 3 to 5. |
| | In some cases, R² is a C₁ alkyl group or a C₂-C₆ saturated linear or branched alkyl |
| | group, and w is an integer from 2 to 14. |
| | In some cases, R² is a C₁-C₂ alkyl group and w is an integer from 2 to 6. |
| | In some cases, n is an integer selected from 0-500. |
| | In some cases, n is an integer selected from 0-100. |
| | In some cases, n is an integer selected from 5-50. |
| | In some cases, m is an integer selected from 0-500. |
| | In some cases, m is an integer selected from 0-100. |
| | In some cases, m is an integer selected from 5-50. |
| 13 | 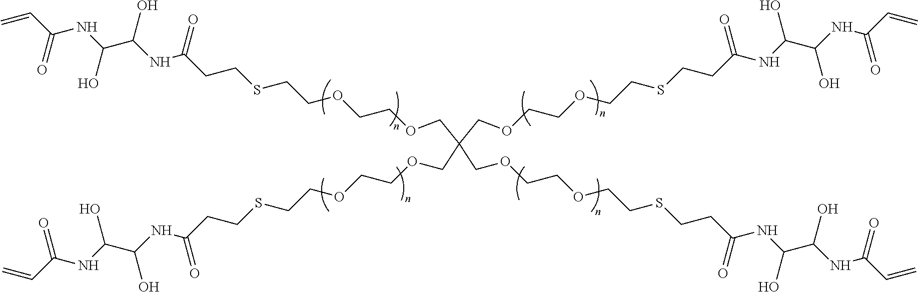 |
| | In some cases, n is an integer selected from 0-500. |
| | In some cases, n is an integer selected from 0-100. |
| | In some cases, n is an integer selected from 5-50. |
| 14 | 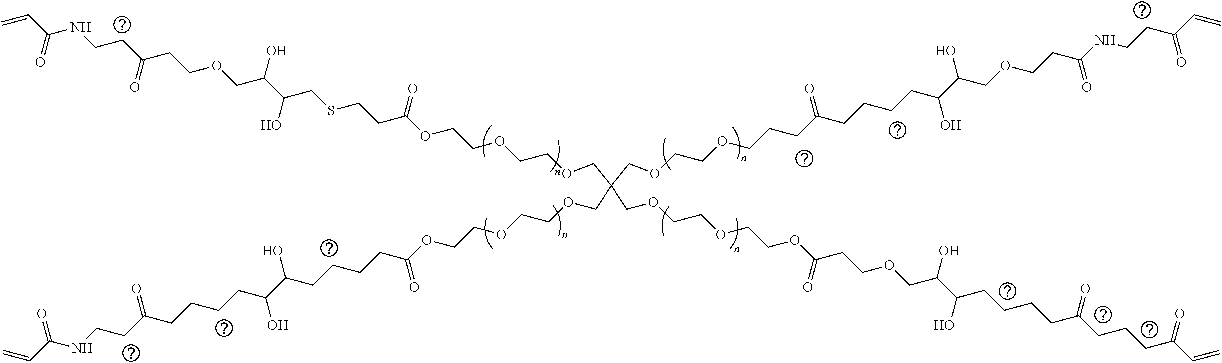 |
| | In some cases, n is an integer selected from 0-500. |
| | In some cases, n is an integer selected from 0-100. |
| | In some cases, n is an integer selected from 5-50. |
| 15 | 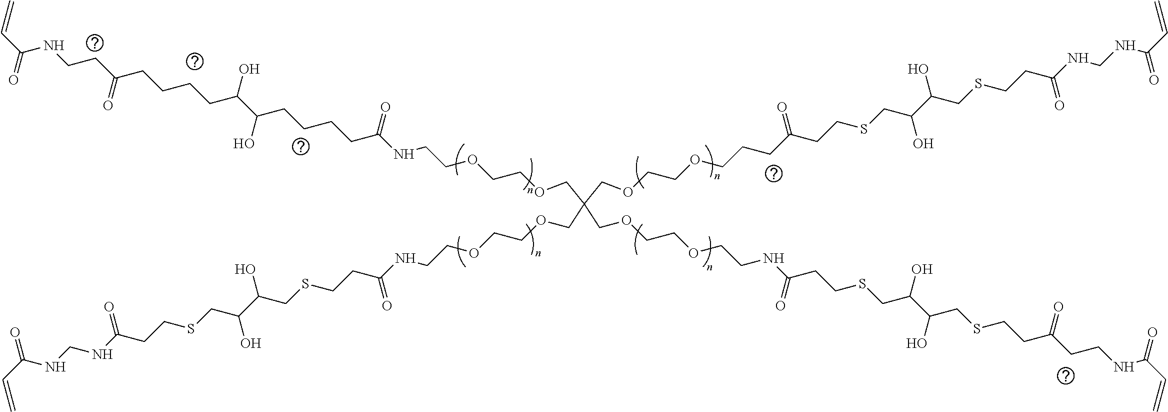 |
| | In some cases, n is an integer selected from 0-500. |
| | In some cases, n is an integer selected from 0-100. |
| | In some cases, n is an integer selected from 5-50. |
| 16 | 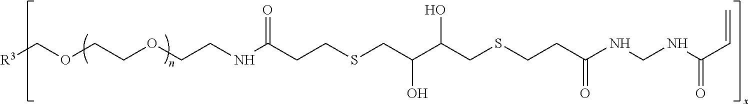 |
| | wherein R³ is: |
| | i) a C₁ alkyl group, |
| | ii) a C₂-C₁₈ linear or branched saturated alkyl group, or |
| | iii) a C₃-C₈ cyclic saturated alkyl group substituted with 0-4 |
| | independently selected C₁-C₃ alkyl groups; |
| | wherein R³ is substituted with x instances of |
| | 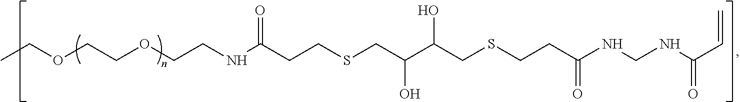 |
| | the remaining substituents on R³ are hydrogen, x is an integer from 2 to 32, and n is an |
| | integer selected from 0-500. |
| | In some cases, x is an integer from 2 to 10. |
| | In some cases, x is an integer from 2 to 6. |
| | In some cases, x is an integer from 3 to 5. |
| | In some cases, R³ is a C₁ alkyl group or a C₂-C₆ saturated linear or branched alkyl |
| | group, and x is an integer from 2 to 14. |
| | In some cases, R³ is a C₁-C₂ alkyl group and x is an integer from 2 to 6. |
| | In some cases, n is an integer selected from 0-500. |
| | In some cases, n is an integer selected from 0-100. |
| | In some cases, n is an integer selected from 5-50. |
| 17 | 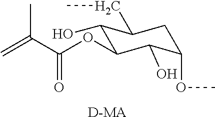 |
| 18 | 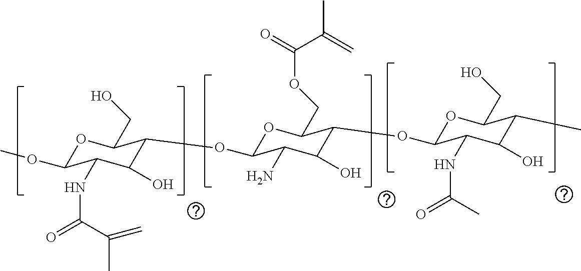 |
| | In some cases, n is an integer selected from 0-500. |
| | In some cases, n is an integer selected from 0-100. |
| | In some cases, n is an integer selected from 5-50. |
| | In some cases, m is an integer selected from 0-500. |
| | In some cases, m is an integer selected from 0-100. |
| | In some cases, m is an integer selected from 5-50. |
| | In some cases, p is an integer selected from 0-500. |
| | In some cases, p is an integer selected from 0-100. |
| | In some cases, p is an integer selected from 5-50. |
| 19 | 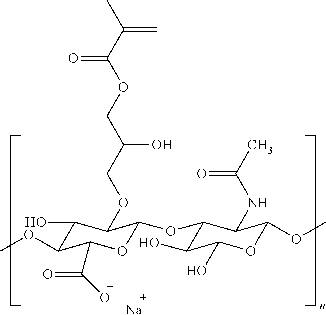 |
| | In some cases, n is an integer selected from 0-500. |
| | In some cases, n is an integer selected from 0-100. |
| | In some cases, n is an integer selected from 5-50. |
| 20 | 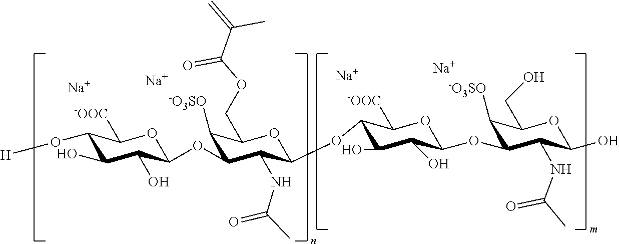 |
| | In some cases, n is an integer selected from 0-500. |
| | In some cases, n is an integer selected from 0-100. |
| | In some cases, n is an integer selected from 5-50. |
| | In some cases, m is an integer selected from 0-500. |
| | In some cases, m is an integer selected from 0-100. |
| | In some cases, m is an integer selected from 5-50. |
| | In some cases, m is an integer from 0-10. |
| 21 | 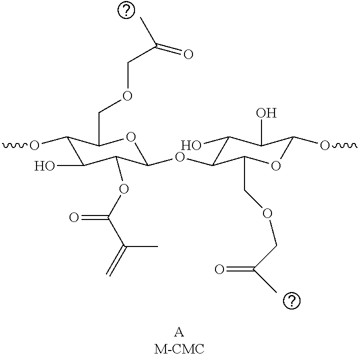 |
| 22 | 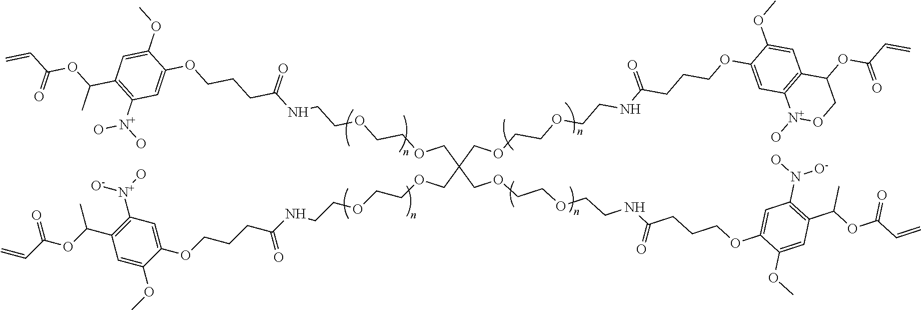 |
| | In some cases, n is an integer selected from 0-500. |
| | In some cases, n is an integer selected from 0-100. |
| | In some cases, n is an integer selected from 5-50. |
| 23 | 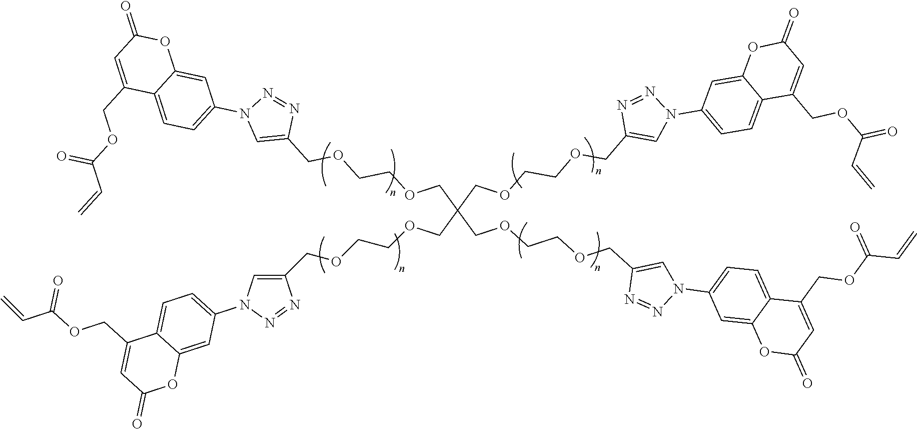 |
| | In some cases, n is an integer selected from 0-500. |
| | In some cases, n is an integer selected from 0-100. |
| | In some cases, n is an integer selected from 5-50. |

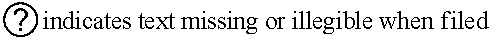

A polymer precursor can further comprise additional reagents that affect polymerization and polymer matrix properties. As examples, a polymer precursor can include a crosslinker, a porogen, a viscosity-modifying agent, an acid, a base, a catalyst, a salt, a photoinitiator, or a combination thereof.

As used herein, the term “crosslinker” denotes a species with a greater number of polymerizable groups than are required to generate a linear polymer chain. For example, a crosslinker can contain two or more ethylenically unsaturated groups or three or more reactive centers for bifunctional polymer synthesis (e.g., the three methoxy groups of trim eth oxybenzene in the context of polyester synthesis). Crosslinkers are thus generally configured to generate a nonlinear polymer network upon polymerization.

As used herein, the term “porogen” can denote a species that modulates the porosity of a polymer matrix but does not incorporate into the polymer matrix during polymerization. Porogens typically diffuse out of polymer matrices following polymerization, leaving pores in the regions that they occupied. Porogen size, concentration, hydrophobicity, and hydrophilicity can thus influence pore density and pore size in polymer matrices. Examples of porogens consistent with the present disclosure include particles (e.g., polymeric, ceramic, metal, metal oxide, or hydrogel particles), polymers such as polyethylene glycol and alginate, and vesicles such as liposomes or micelles.

As used herein, the term “photoinitiator” can denote a species that generates a radical upon photoexcitation. In many cases, a photoinitiator included in a polymer precursor formulation is a type I photoinitiator, that is a molecule that generates radicals through intramolecular cleavage (e.g., homolysis) upon photoexcitation, or a type II photoinitiator, that is a molecule that abstract an electron or hydrogen atom from a co-initiator following photoexcitation. Examples of photoinitiators utilizable in the present methods include acetophenone, anisoin, anthraquinone, anthraquinone-2-sulfonic acid, benzil, benzoin, benzophenone, 3,3′,4,4′-benzophenonetetracarboxylic dianydride, 4-benzoylbiphenyl, 2-benzyl-2-(dimethylamino)-4′-morpholinobutyrophenone, dibenzosuberenone, 2,2-diethoxyacetophenone, 2-ethylanthraquinone, ferrocene, 2-isopropylthioxanthone, lithium phenyl (2,4,6-trimethylbenzoyl) phosphinate, methyl-2-benzoylbenzoate, and thiooxanthen-9-one.

In some embodiments, the generation of a polymer matrix within said fluidic device comprises exposing the one or more polymer precursors to an energy source. In some embodiments, the energy source is a light generating device. In some embodiments, the light generating device generates light at 350 nm to 800 nm. In some embodiments, the light generating device generates light at 350 nm to 600 nm. In some embodiments, the light generating device generates light at 350 nm to 450 nm. In some embodiments, the light generating device generates UV light. In some embodiments, the generation of the polymer matrix comprises between about 1 and 3 seconds of illumination, between about 1 and 5 seconds of illumination, between about 1 and 10 seconds of illumination, between about 1 and 15 seconds of illumination, between about 1 and 20 seconds of illumination, between about 1 and 30 seconds of illumination, between about 1 and 50 seconds of illumination, between about 3 and 5 seconds of illumination, between about 3 and 10 seconds of illumination, between about 3 and 15 seconds of illumination, between about 3 and 20 seconds of illumination, between about 3 and 30 seconds of illumination, between about 3 and 50 seconds of illumination, between about 5 and 10 seconds of illumination, between about 5 and 15 seconds of illumination, between about 5 and 20 seconds of illumination, between about 5 and 30 seconds of illumination, between about 5 and 50 seconds of illumination, between about 10 and 20 seconds of illumination, between about 10 and 30 seconds of illumination, between about 10 and 50 seconds of illumination, between about 20 and 30 seconds of illumination, or between about 20 and 50 seconds of illumination. In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid crystal based.

Optionally, a first chamber of the one or more chambers can be disposed inside of a second chamber of the one or more chambers. This design can be utilized to separately partition two species (e.g., a cell and a reagent or two cells) within close proximity. This design can also be used to control the timing with which two species are contacted. For example, a method can include forming a first chamber around a cell, flowing a bead (or other assay reagent incapable of diffusing into the first chamber) adjacent to the cell, forming a second chamber surrounding the first chamber and enclosing the bead, and selectively degrading the first chamber to allow the cell and bead to come into contact within the second chamber.

In some embodiments, a functional group can be coupled to one or more chambers. Some non-limiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized chamber may be used to capture a biomolecule enclosed therein, thereby trapping the biomolecule in proximity to a biological component (e.g., a cell) enclosed within the chamber. The biomolecule may be produced by the biological component (e.g., a facet of a cell secretome). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

With continued reference to FIG. 2A, the polymer matrix 208, 209, or at least a portion of the polymer matrix 208, 209, may be coupled to the first surface 201, the second surface 202, or both surfaces 201, 202. In certain embodiments, the polymer matrix, or at least a portion of the polymer matrix, may be coupled to a third surface, a fourth surface, a fifth surface, etc., as appropriate. In various embodiments, the polymer matrix 208, 209 may extend from the first surface 201 to the second surface 202 (e.g., through at least a portion of a lumen of the channel 200 or a cavity of a chamber) such that the polymer matrix surrounds, or substantially surrounds, the biological component 50. In some embodiments, two or more biological components (e.g., biological components 50, 51 of FIG. 2C) that are in close physical proximity may be separated (e.g., by agitating or shaking the fluidic device). The fluidic device may be agitated or shaken by physical movement, use of a sonic pulse, changing a flow in the channel, or any other suitable method of agitation. A polymer matrix may then be formed that surrounds (or partially surrounds) the biological components that are separated. FIG. 2B shows polymer matrices 208, 209 formed surrounding the biological component 50 after being separated from the biological component 51. FIG. 2C shows a process, according to various embodiments, of separating the two biological components 50, 51, which are in close proximity. That is, by agitating or shaking the fluidic device the biological components 50, 51 can be separated. In some embodiments, separation of the biological components is achieved through fluidic pressure, flow pulsation, dielectrophoresis, optothermal flow, or some combination thereof. In some cases, separation of the biological components is achieved through acoustic vibration. FIG. 2C also shows a polymer matrix being formed to generate a compartment 222 surrounding the biological component 50 after the separation of the biological components 50, 51.

A fluidic device disclosed herein can include a detector that is configured to detect one or more locations of one or more biological components contained within a channel. In certain embodiments, the energy source 203 can comprise, be coupled to, or be in communication with a detector that detects, or is configured to detect, a location of a biological component in the fluidic device. In various embodiments, a mask may be generated using an image obtained from at least a portion of the fluidic device. The mask may allow or permit the energy source 203 to emitting energy in or toward one or more locations or positions where one or more biological components are present on or adjacent the first surface 201. The mask may inhibit or prevent the energy source 203 from emitting energy in or toward one or more locations or positions where one or more biological components are present on or adjacent the first surface 201. In some embodiments, the image may be obtained from a camera (e.g., a digital camera, fluorescent imaging camera, etc.). In some embodiments, the camera maybe coupled to, connected to, or in communication with the energy source 203. For example, the camera (not shown) may be in electrical communication with the energy source 203. In some embodiments, the energy source 203 may comprise the camera. In various embodiments, the energy source 203 may comprise a microscope (e.g., a fluorescence microscope, a confocal microscope, lens-free imaging system, a transmission electron microscopy (TEM), a scanning electron microscope (SEM), etc.). The microscope may be used to detect one or more positions of one or more biological components (e.g., in combination with the detector).

In some embodiments, one or more chambers has sufficiently large pores to allow movement or transfer of a reagent (e.g., an enzyme, a chemical compound, a small molecule, an antibody, etc.) therethrough. Simultaneously, one or more chambers can have sufficiently small pores to allow movement or transfer of a reagent and/or biological component (e.g., DNA, RNA, a protein, a cell, etc.). In some embodiments, the pores have a diameter from 5 nm to 100 nm. In some embodiments, the pores have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores have a diameter smaller than 5 nm.

In particular embodiments, pores of the one or more chambers are formulated to encapsulate sufficiently large genetic material, nucleic acids with greater than 300 base pairs, but to allow smaller materials, such as reverse transcriptases and 50 base pair nucleic acid primers to pass through the pores, thereby passing in and out of the hydrogel structures. In some embodiments, the pore size of the hydrogel structures is tuned by varying the ratio of the concentrations of polymer precursors to the concentration of crosslinkers, varying pH, salt concentrations, temperature, light intensity, and the like. In some embodiments, the average diameter of pores of a chamber prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater. In some embodiments, DNA or RNA retained have lengths that are sequencable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm.

The pore sizes of the one or more chambers may be modulated using a chemical reagent, or by applying heat, electrical field, light, or another suitable stimulus. In other words, a chamber may comprise a tunable property (e.g., the pore size). In some cases, one or more chambers comprises a thermoresponsive or temperature-responsive polymer. A thermoresponsive polymer (e.g., poly(N-isopropylacrylamide) (NIPAAM)) may phase separate from a solution upon heating or upon cooling (e.g., polymer showing lower critical solution temperature (LCST) or upper critical solution temperature (UCST)). The polymer matrix may comprise polymer which may collapse at high temperature in order to, for example, control the pore size of the hydrogel or polymer matrix. Non-limiting examples of thermoresponsive polymers that may be used to form hydrogel/polymer matrix with tunable properties may include Poly(N-vinyl caprolactam), Poly(N-ethyl oxazoline), Poly(methyl vinyl ether), Poly(acrylic acid-coacrylamide), or a combination thereof. A change in temperature may enlarge or contract average pore size in the polymer matrix to allow selected molecules, such as a nucleic acid molecule, a protein, or any biomolecule or molecule smaller than the adjusted pore size to be released from a hydrogel chamber.

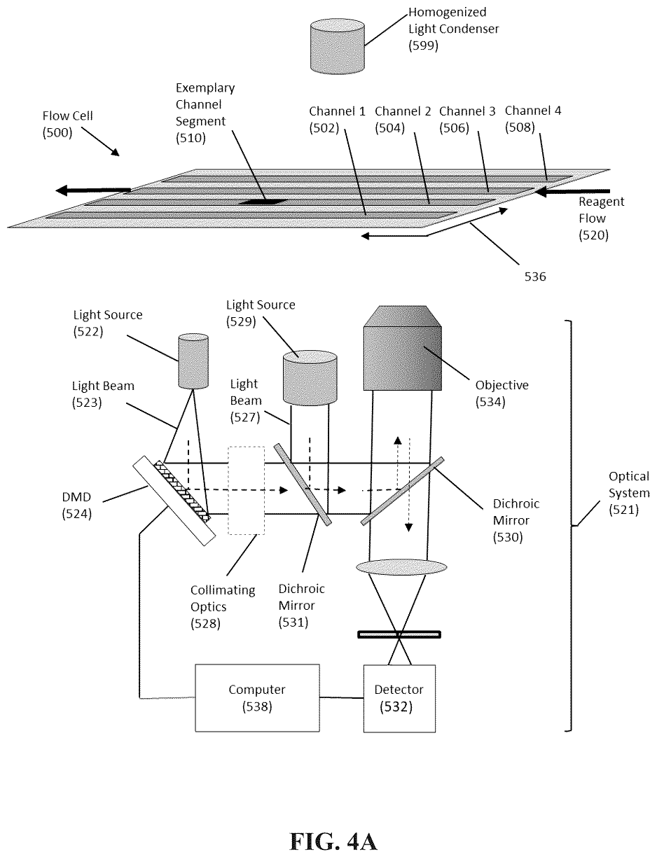

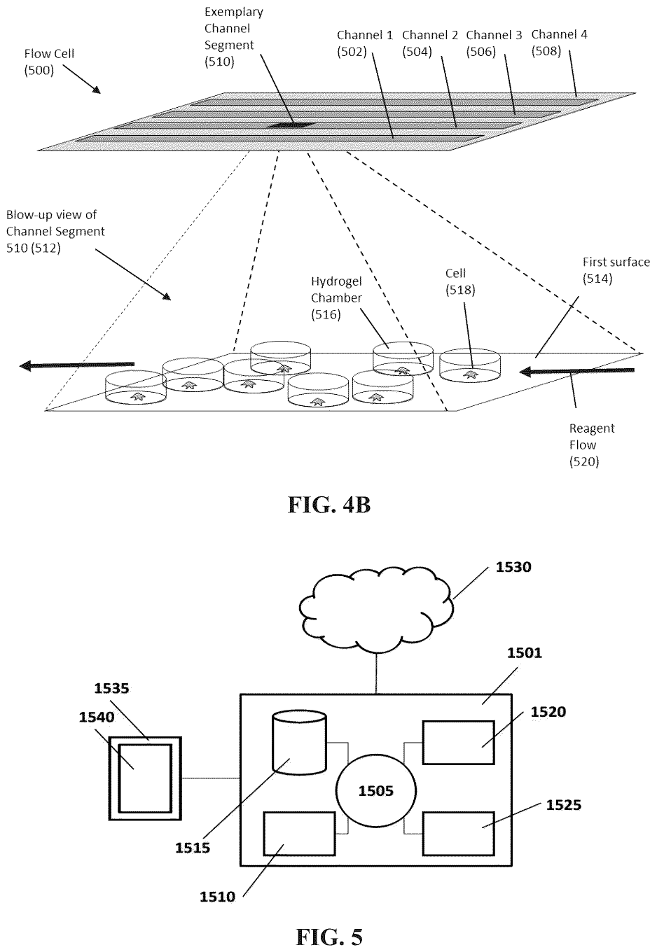

FIG. 4A is an example system for carrying out the above method. Flow cell (500) is a component of a fluidic device that provides channels for carrying out a variety of assays and liquid handling components under programmable control for delivering samples and reagents to the channels. In this illustration, four channels (502, 504, 506, and 508) are shown. However, as detailed elsewhere herein, systems of the present disclosure can utilize flow cells with fewer or greater numbers of channels.

The system of FIG. 4A includes an optical system (521) for photosynthesizing chambers at locations of cells or other analytes in the channels (502, 504, 506, and 508) of the flow cell (500) and for collecting images and other optical signals. The optical system (521) includes a light source (522) that generates a light beam (523) of appropriate wavelength light (e.g. UV light) for synthesizing chambers (e.g., hydrogel chambers) in the flow cell (500). The light beam (523) that passes through an appropriate photo-mask or beam-shaping or beam steering (Galvo) system (524) for shaping a beam to synthesize a desired structure or structures in a channel. In some embodiments, this beam shaping system (524) includes a digital micromirror device (DMD). In other embodiments, a physical photo-mask may be employed. Reflected light from DMD (524) is shaped using conventional optics, e.g. collimating optics (528), and is directed through objective lens system (534) into channel 2 segment (510). In exemplary embodiments, the light is directed by one or more dichroic mirrors (530 and 531).

Chamber position, shape and polymer matrix wall thickness is determined at least in part from cell position information determined from images collected by detector (532). Objective (534) and flow cell (500) move relative to one another in the xy-directions (536) to photosynthesize chambers at any position in any of the channels. In some embodiments, the flow cell (500) moves and optical system (521) is stationary. The system may utilize light from a light source (599), such as a homogenized light condenser, that is positioned on an opposite side of the flow cell as the optical system and directs light through the flow cell (500) to the objective (534). To achieve this functionality, the light source positioned on the opposite side of the flow cell (599) can be configured to move in tandem with the optical system (521), or the light source (599) and optical system can be stationary and the flow cell (500) can be moved to the region illuminated by the light source (599) and from which light is collected by the objective (534). In some embodiments, objective (534) may also direct light beam (527) from light source (529) to targets, such as cells, on first surface (514) and collect optical signals, such as fluorescent signals, from assays taking place on first surface (514). Optical signal collection can also be carried out with a separate objective. Information collected by detector (532), particularly cellular positions in their respective channels, is employed by computer (538) and/or subsidiary controllers to direct DMD (524) and translation devices controlling the relative positions of objective (534) and flow cell (500) to synthesize hydrogel chambers of the appropriate shape and size at the appropriate locations.

FIG. 4B provides a blown-up view of the exemplary channel segment (510) of the flow cell of FIG. 4A. On first surface (514) of channel 2 (504) a plurality of cells, e.g. (518), are each enclosed by a hydrogel chamber, e.g. (516). In some embodiments, the porosity of polymer matrix walls of the hydrogel chambers is selected to be impermeable to the cells, but permeable to assay reagents. Thus, reagents may be introduced to, and removed from, the interiors of the hydrogel chambers by flowing (520) them through the channels, but cells (518) are retained in hydrogel chamber (516).

One of ordinary skill in the art would recognize that optical systems with different configurations than those of FIGS. 4A and 4B may be employed for carrying out these functions. In some embodiments, a plurality of DMD-objective subsystems for synthesizing hydrogel structures may be employed to increase the speed of synthesis by synthesizing multiple structures simultaneously.

The present disclosure provides computer systems that are programmed to implement methods of the disclosure. FIG. 5 shows a computer system 1501 that may be programmed or otherwise configured to perform methods described herein. The computer system 1501 can regulate various aspects of the present disclosure, such as, for example, identifying a biological component, detecting a barcode, generating a spatial modulating element (e.g., a mask), providing energy from an energy source, or detecting or measuring a local parameter using a sensor. The detector may be a camera (e.g., a fluorescent camera), such as a charged coupled device (CCD) camera capable of collecting optical signals and position information from a plurality of sources distributed over a planar region. The computer system 1501 can be an electronic device of a user or a computer system that may be remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet or an intranet and/or extranet that may be in communication with the Internet. The network 1530 in some cases may be a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit may be an application specific integrated circuit (ASIC).

The storage unit 1515 can store files, such as drivers, libraries, and saved programs. The storage unit 1515 can store user data, e.g., user preferences and user programs. The computer system 1501 in some cases can include one or more additional data storage units that are external to the computer system 1501, such as located on a remote server that may be in communication with the computer system 1501 through an intranet or the Internet.

The computer system 1501 can communicate with one or more remote computer systems through the network 1530. For instance, the computer system 1501 can communicate with a remote computer system of a user (e.g., a laptop, a personal computer, a tablet, or a mobile phone). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, an image of a biological component, a barcode, a signal or measurement of a local parameter. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, identify a biological component, detect a barcode, generate a spatial modulating element (e.g., a mask), provide energy from an energy source, detect or measure a local parameter using a sensor, etc.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

### Example 1

Interstitial Fibroblast Removal from a Flow Cell Channel

This example is directed to interstitial cell removal from a flow cell channel with an adhesion inhibitor. The flow cell channel was prepared with first cells enclosed in hydrogel chambers and second cells (‘interstitial cells’) disposed outside of the hydrogel chambers. Prior to cell loading and analysis, phosphate-buffered saline (PBS) containing 50 μg/mL fibronectin was incubated in the channel for about thirty minutes to coat flow cell surfaces with fibronectin. The channel was flushed with PBS buffer to wash away suspended fibronectin.

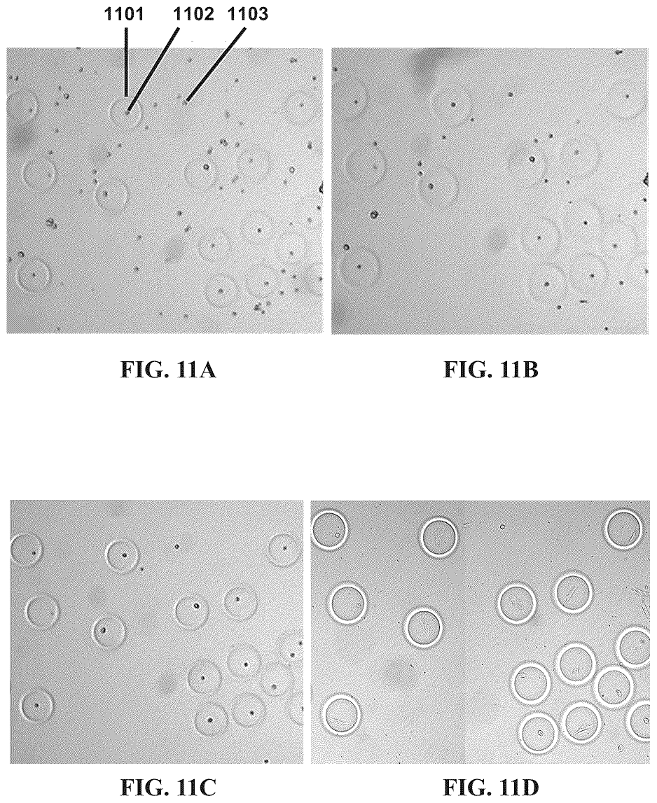

Fibroblasts were then loaded into the channel in media containing photopolymerizable hydrogel precursors and 2.5 mg/mL RGD peptide intended to limit cell binding to the fibronectin coating. Cylindrical hydrogel chambers were formed around a subset of fibroblasts in the channel using a digital micromirror device (DMD) to project light to specified regions of the channel. An exemplary image of a portion of the channel following chamber synthesis is shown in FIG. 11A, wherein exemplary chambers, (1101), enclosed fibroblasts (1102), and non-enclosed (“interstitial”) fibroblasts (1103) are labeled. FIGS. 11B-D, which were taken at later time points, cover the same field as FIG. 11A. At this stage, the fibroblasts exhibited spherical morphologies, suggesting that they were suspended or poorly adhered to the surface.

The channel was then washed with Ca²⁺ and Mg²⁺-free 200 μL PBS, more than 3-times the volume of the channel. An image of the channel following this wash is shown in FIG. 11B, showing that a majority of interstitial fibroblasts were removed during this step. It was hypothesized that the remaining fibroblasts were at least partially adhered to top or bottom surfaces of the channel

The channel was then loaded with 5 mM EDTA in PBS, incubated for 5 minutes at 37° C., and then washed twice with Ca²⁺ and Mg²⁺-free 200 μL PBS. FIG. 11C provides an image of the channel following these treatments. The interstitial space contains few fibroblasts, suggesting that EDTA treatment and further PBS washes were effective for removing adhered fibroblasts from the channel.

Following interstitial fibroblast removal, cell culture medium was added to and incubated within the channel. An image of the flow cell following this incubation period is shown in FIG. 11D, wherein the enclosed fibroblasts exhibit spread-out morphologies and lower optical densities indicating adherence to the fibronectin-coated surface.

### Example 2

### Interstitial Cell Removal Using a Detachment Reagent

This example is directed to interstitial cell removal with a cell detachment reagent. Two fluidic device channels were coated with fibronectin by incubating 50 μg/mL fibronectin solution in the channels at room temperature for 30 minutes and then washing the channels with PBS.

Dendritic cells were prepared in IDME media at a density of 10,000 cells/μL or 12,000 cells/μL. The cells were then diluted with a polymer precursor to a final density of 1,000 cells/μL or 1,200 cells/μL and loaded into the fluidic device channels. Hydrogel chambers were formed around select cells before overnight incubation and complete cell adhesion and spreading.

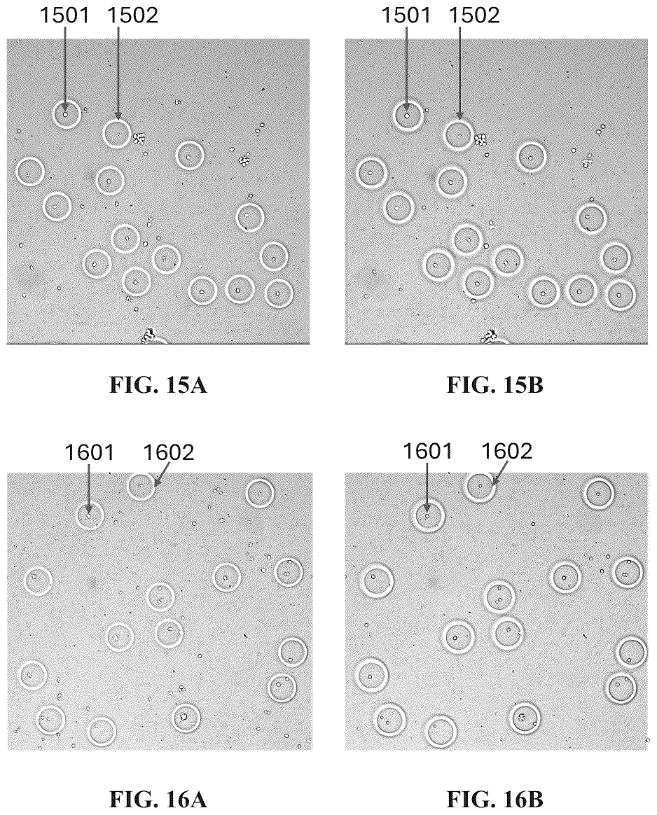

The fluidic device channel loaded with 1,000 cells/μL was then washed with room temperature, pH 7.4 phosphate-buffered saline (PBS) followed by media. Brightfield images of select chambers within the channel before and after the wash steps are shown in FIG. 15A-B, respectively. Within these figures, representative cells and chambers are indicated with the labels 1501 and 1502, respectively. As can be seen in these images, only a minor fraction of interstitial (non-chamber-enclosed) cells were removed during the wash steps.

The fluidic device channel loaded with 1,200 cells/μL was washed with pH 7.4 PBS followed by 5 mM EDTA (incubated for 5 minutes at 37° C.) and finally pH 7.4 PBS. Brightfield images of select chambers within the channel before and after the wash steps are shown in FIG. 16A-B, respectively. Within these figures, representative cells and chambers are indicated with the labels 1601 and 1602, respectively. These images show that the majority of interstitial (non-chamber-enclosed) cells were removed during the wash steps.

### Example 3

### Removal of Interstitial Cells From a Non-Adherent Support-Coated Fluidic Device

This example is directed to interstitial cell removal from a fluidic device in which portions of the bottom surface were coated with an adherent support and portions of the bottom surface were coated with a non-adherent support. Prior to fluidic device fabrication, glass slides were coated with a PEG-grafted poly-l-lysine polymer (PLL-g-PEG). Surfaces of the slides were activated for 10 minutes in air plasma. The surfaces were then submerged in 0.1 mg/mL PLL-g-PEG for one hour at room temperature to coat the surfaces with PLL-g-PEG. The slides were then washed one time with PBS and water, dried under nitrogen, and stored under vacuum.

Portions of one of the slides were then coated in poly-d-lysine (PDL). 140 μL droplets containing 0.1 mg/mL PDL were spotted onto discrete sites in a grid-pattern along the surface. The slide was rinsed with deionized water, dried, and stored at 4° C. under vacuum.

The fluidic device was then fabricated by bonding the slide with the PDL-spotted surface to a PLL-g-PEG-coated slide (that had not been spotted with PDL) with a double-sided adhesive spacer. The PDL-spotted surface constituted the bottom surface of the fluidic device. The double-sided adhesive spacer included multiple cut-out regions that, in combination with the slides, defined channels of the fluidic device.

The PDL-coated portions of the surface were then further coated with fibronectin. The fluidic device was filled with a room temperature solution containing 25 μg/mL fibronectin. The fibronectin solution was incubated in the fluidic device for 30 minutes, and was then removed by flushing the fluidic device with deionized water. The fibronectin adhered to the PDL-coated portions of the bottom surface and did not adhere to portions of the surfaces with exposed PEG (i.e., portions of the surfaces that did not contain PDL).

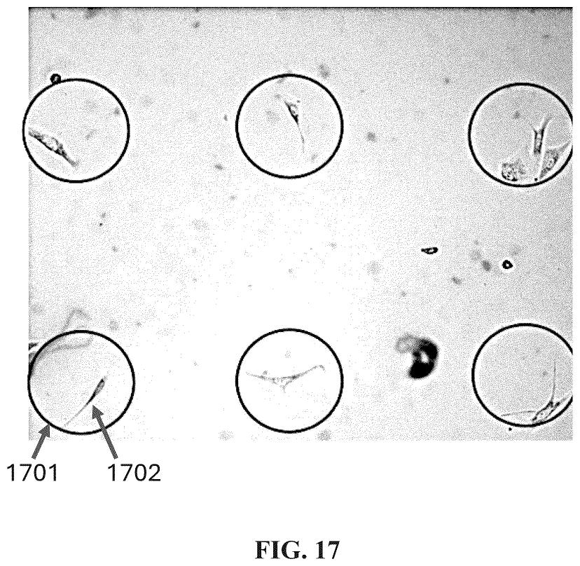

Fibroblasts (IMR-90 cells) were loaded into the fluidic device and incubated at 37° C. overnight. The fluidic device was washed with media to remove non-adhered fibroblasts, and the fluidic device was imaged using brightfield imaging at 10× magnification. A representative image of cells in the fluidic device are shown in FIG. 17. Within this figure, the fibronectin-coated spots are indicated with dark circles, one of which is labeled 1701 for reference. A representative cell is labeled 1702. As can be seen in this image, cells were present within the fibronectin-coated regions of the fluidic device surface and were not present within the non-adherent support-coated portion of the fluidic device surface. Cells within the fibronectin-coated regions exhibited flattened, spread-out morphologies, indicating that the cells were in adherent states.

What is claimed is:

1. A method for separating a plurality of cells comprising:
inputting the plurality of cells into a channel of a fluidic device;
synthesizing one or more chambers in the channel to enclose a first cell of the plurality of cells and not enclose a second cell of the plurality of cells; and
removing the second cell from the channel,
wherein the removing comprises contacting the second cell with a cell detachment reagent that detaches the second cell from a first surface of the channel or from a cell adherent support coupled to the first surface of the channel, and
wherein the first cell remains enclosed in the one or more chambers during the removing.

2. The method of claim 1, wherein prior to the removing, the second cell is adhered to the first surface of the channel or to the cell adherent support coupled to the first surface of the channel.

3. The method of claim 2, wherein the contacting causes the second cell to detach from the first surface of the channel or from the cell adherent support coupled to the first surface of the channel.

4. The method of claim 1, wherein the removing causes the first cell to become suspended within a chamber of the one or more chambers.

5. The method of claim 1, further comprising incubating the first cell following the removing, wherein during the incubating, the first cell binds or adheres to the first surface of the channel or to the cell adherent support coupled to the first surface of the channel.

6. The method of claim 1, wherein the one or more chambers co-enclose a capture element with the first cell.

7. The method of claim 6, wherein the one or more chambers co-enclose the capture element and the cell adherent support with the first cell, and wherein:
(i) the cell adherent support is coupled to the first surface of the channel and the capture element is coupled to a second surface of the channel opposite the first surface of the channel; or
(ii) the cell adherent support and the capture element are coupled to the first surface of the channel.

8. The method of claim 7, wherein the cell adherent support is adjacent to the capture element on the first surface of the channel.

9. The method of claim 6, wherein the capture element comprises an oligonucleotide.

10. The method of claim 9, wherein the oligonucleotide comprises an mRNA capture sequence.

11. A method for separating a plurality of cells comprising:
inputting the plurality of cells into a channel of a fluidic device;
contacting the plurality of cells with an adhesion inhibitor;
synthesizing one or more chambers in the channel to enclose a first cell of the plurality of cells and not enclose a second cell of the plurality of cells; and
removing the second cell from the channel, wherein the first cell remains enclosed in the one or more chambers during the removing.

12. The method of claim 11, wherein:
(i) the contacting is prior to the inputting;
(ii) the contacting is subsequent to the inputting; or
(iii) the inputting comprises loading the adhesion inhibitor into the channel with the plurality of cells.

13. The method of claim 11, wherein the adhesion inhibitor is loaded into the channel with one or more polymer precursors of the one or more chambers, and wherein the synthesizing comprises forming one or more walls of the one or more chambers using the one or more polymer precursors.

14. The method of claim 11, wherein the adhesion inhibitor diminishes a binding affinity of at least a subset of the plurality of cells to a first surface of the channel or a cell adherent support coupled to the first surface of the channel.

15. The method of claim 11, wherein the adhesion inhibitor diminishes an integrin binding affinity of the plurality of cells of the subset of the plurality of cells.

16. The method of claim 11, wherein the adhesion inhibitor binds to the plurality of cells or the subset of the plurality of cells.

17. The method of claim 15, wherein the adhesion inhibitor comprises an antibody, actinin, collagen, fibrinogen, fibronectin, gelatin, ICAM-1, ICAM-2, laminin, osteopontin, paxillin, poly-l-lysine (PLL), poly-d-lysine (PDL), poly-l-ornithine, talin, VCAM-1, vinculin, vitronectin, a cell adherent peptide, or a combination thereof.

18. The method of claim 11, wherein the adhesion inhibitor comprises a metal chelator.

19. The method of claim 11, further comprising releasing the adhesion inhibitor from the first cell.

20. The method of claim 19, wherein the releasing comprises contacting the first cell with a cell detachment reagent, sonicating the fluidic device, ultrasonicating the fluidic device, or a combination thereof.


SEQUENCE LISTING (10 sequences)

SEQ ID NO: 1 - protein, 6 aa, synthetic construct
  SITE 3: note = Hydroxyproline
GFXGER

SEQ ID NO: 2 - protein, 5 aa, synthetic construct
YIGSR

SEQ ID NO: 3 - protein, 5 aa, synthetic construct
GRGDS

SEQ ID NO: 4 - protein, 13 aa, synthetic construct
CKKQRFRHRNRKG

SEQ ID NO: 5 - protein, 4 aa, synthetic construct
KRSR

SEQ ID NO: 6 - protein, 5 aa, synthetic construct
VPGIG

SEQ ID NO: 7 - protein, 7 aa, synthetic construct
MNYYSNS

SEQ ID NO: 8 - protein, 6 aa, synthetic construct
CSVTCG

SEQ ID NO: 9 - protein, 7 aa, synthetic construct
GFRGDGQ

SEQ ID NO: 10 - protein, 7 aa, synthetic construct
FLPASGL